(19) United States
(12) Patent Application Publication
(10) Pub. No.: US 2025/0163069 A1
(43) Pub. Date: May 22, 2025

(54) HETEROCYCLIC DERIVATIVE, AND PHARMACEUTICAL COMPOSITION AND APPLICATION THEREOF

(71) Applicant: SUZHOU YUANZHI PHARMACEUTICAL TECHNOLOGY CO., LTD., Suzhou (CN)

(72) Inventors: Li Chen, Suzhou (CN); Qin Cao, Suzhou (CN); Libin Gan, Suzhou (CN)

(21) Appl. No.: 18/841,062

(22) PCT Filed: May 18, 2023
(86) PCT No.: PCT/CN2023/095025

§ 371 Date: Aug. 23, 2024

(30) Foreign Application Priority Data
Jun. 8, 2022 (CN) 202210645757.0

(51) Int. Cl. C07D 487/08; A61K 31/517; A61K 45/06; A61P 31/22; C07D 401/14; C07D 403/04; C07D 405/14

(52) U.S. Cl. CPC C07D 487/08; A61K 31/517; A61K 45/06; A61P 31/22; C07D 401/14; C07D 403/04; C07D 405/14

(57) ABSTRACT

Disclosed in the present disclosure are a heterocyclic derivative represented by formula (I), or a stereoisomer, a pharmaceutically acceptable salt thereof. The heterocyclic derivative or the stereoisomer thereof has high HCMV inhibiting activity, which is increased in potency by 8-40 folds compared with letermovir, and clinically it may provide better protection for patients suffering from HCMV infection after allogeneic hematopoietic stem cell transplantation, kidney transplantation, and lung transplantation.

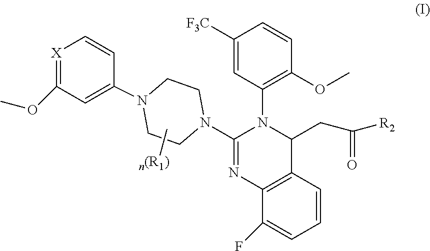

### CROSS-REFERENCE TO RELATED APPLICATIONS

This application is U.S. national phase under 35 U.S.C. § 371 of International Patent Application No. PCT/CN2023/095025, filed on May 18, 2023, which claims priority to Chinese Application No. 202210645757.0, filed on Jun. 8, 2022, the contents of all of which are incorporated by reference in their entirety.

### TECHNICAL FIELD

The present disclosure belongs to the field of medicinal technology, in particular, relates to a heterocyclic derivative and a pharmaceutically composition and application thereof.

### BACKGROUND

Human Cytomegalovirus (HCMV) has double stranded DNA and belongs to the β-subfamily of Herpesviridae family. After HCMV infection, it can remain latent in the host for a long time. When the immune system is weakened or damaged, the latent HCMV virus is reactivated and active infection occurs repeatedly and alternately. HCMV often shows asymptomatic infection in people with normal immune function, but it has a high rate of morbidity and mortality in AIDS patients, organ transplant patients or other immunocompromised populations. HCMV is also a major viral cause of neonatal congenital growth retardation and mental retardation. The standard treatment for HCMV-infected patients is intravenous injection of ganciclovir, foscarnet sodium, cidofovir, or oral administration of valganciclovir. Although the clinical outcomes of these nucleoside drugs are obvious, these treatments result in significant nephrotoxicity and extensive drug resistance, making use of these drugs in recipients of allogeneic hematopoietic cell transplantation more difficult.

Letermovir is a potent viral terminase inhibitor that specifically inhibits the CMV virus terminase complex encoded by CMV genes UL56, UL51, and UL89, preventing reactivation and frequently recurring infection of CMV virus in patients with CMV serum-positive allogeneic hematopoietic stem cell transplantation. Due to that letermovir specifically inhibits the complex enzyme of the virus and does not interact with any enzyme in the human body, it has favorable safety and clinical benefits. After 28 days of treatment, no CMV virus was detected in the patient, and there was no cross resistance with other approved anti-HCMV drugs.

The oral dose of letermovir alone is 480 milligrams once a day. If co-administered with cyclosporine, the QD dosage is adjusted to 240 milligrams. Although it can be rapidly absorbed from the small intestine, the bioavailability of letermovir in hematopoietic cell transplant (HCT) recipients is about 37%. Cyclosporin, as a broad efflux protein (P-gp, MRP-1, BCRP, and LRP) inhibitor, can effectively enhance the bioavailability of letermovir to 85%.

Although the standard treatment DNA polymerase inhibitors, such as valganciclovir, foscarnet sodium or cidofovir, have favorable clinical outcomes, there are serious toxic side effects and extensive drug resistance, and letermovir needs to be combined with cyclosporine, so its potency and dosage need to further optimized. Therefore, in clinical practice, it is necessary to use appropriate administration approaches, and high-potency and low-clinical dose drugs to better meet the clinical needs, and to provide safe, potent, low-toxic, and booster-free anti-HCMV drugs for organ transplant patients.

### SUMMARY OF THE DISCLOSURE

The technical problem to be solved by the present disclosure is to provide a series of heterocyclic derivatives with higher anti-HCMV activity to address the shortcomings and deficiencies of the prior art.

To solve the above technical problem, the present disclosure employs the following technical solution:

A heterocyclic derivative represented by formula (I) or a stereoisomer, a pharmaceutically acceptable salt thereof,

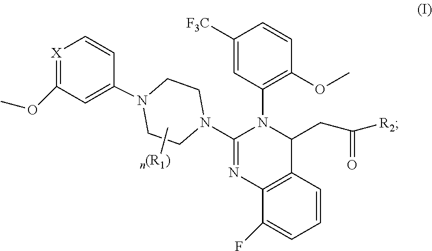

wherein:
R₁ is independently selected from C₁₋₆ alkyl, or two independent R₁ together with the different carbons to which they are attached on the piperazine form a bridged ring;
n is selected from 0, 1, or 2;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy; X is selected from N or —CH—;
When n is 0 and X is —CH—, R₂ is not hydroxyl.

In some embodiments, the disclosed heterocyclic derivative is represented by the following formula (I-a):

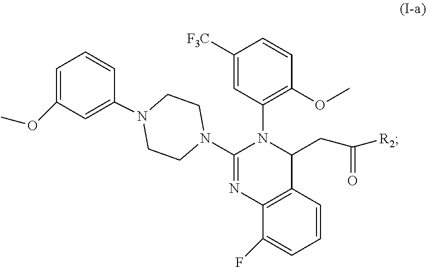

wherein:
R₂ is selected from C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy.

In some embodiments, the disclosed heterocyclic derivative is represented by the following formula (I-b):

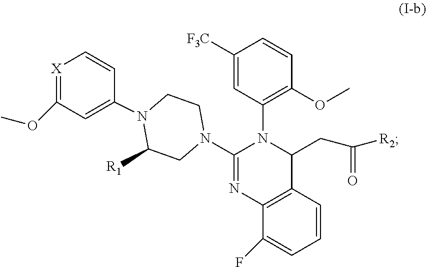

wherein:
R₁ is selected from C₁₋₆ alkyl;
X is selected from N or —CH—;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy.

In some embodiments, the disclosed heterocyclic derivative is represented by the following formula (I-c):

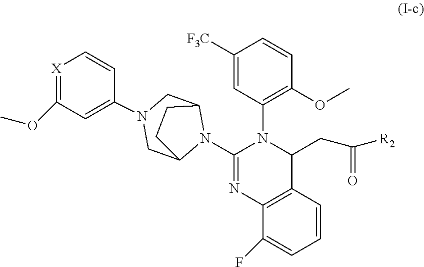

wherein:
X is selected from N or —CH—;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy.

In some embodiments, R₁ is selected from methyl, ethyl or isopropyl.

In some embodiments, R₁ is methyl.

In some embodiments, R₂ is selected from hydroxyl,

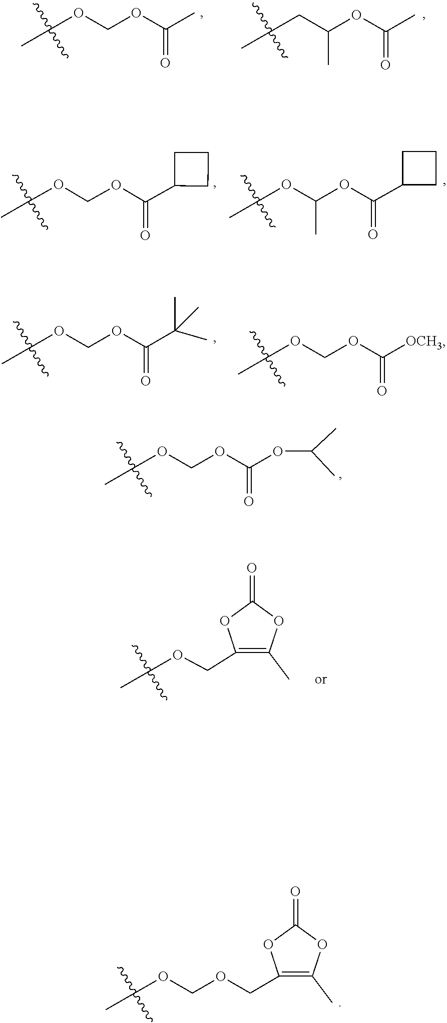

In some embodiments in formula (I-a), R₂ is selected from

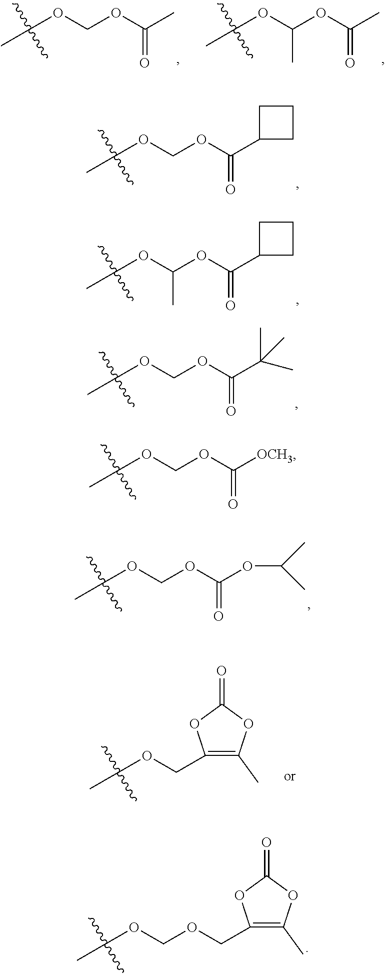

In some embodiments, in formula (I-b) or (I-c), R₂ is selected from hydroxyl,

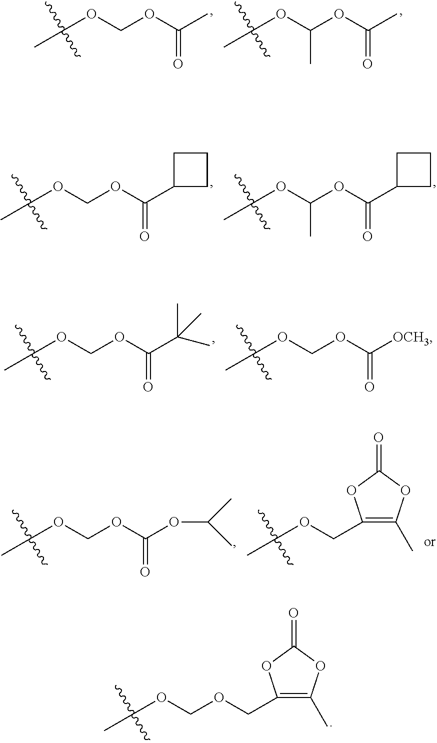

In some embodiments, the heterocyclic derivative or its stereoisomer is selected from the following compounds

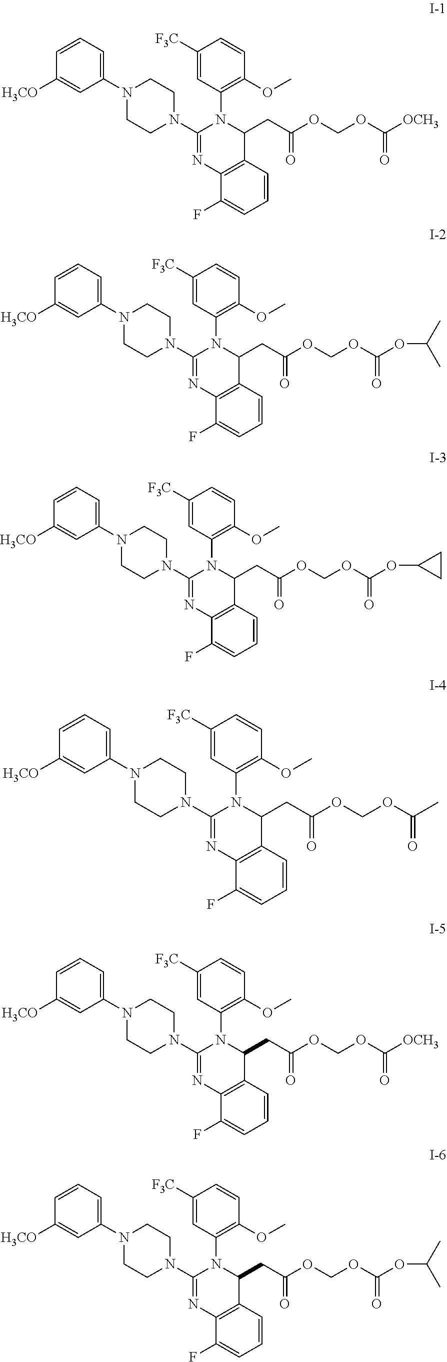

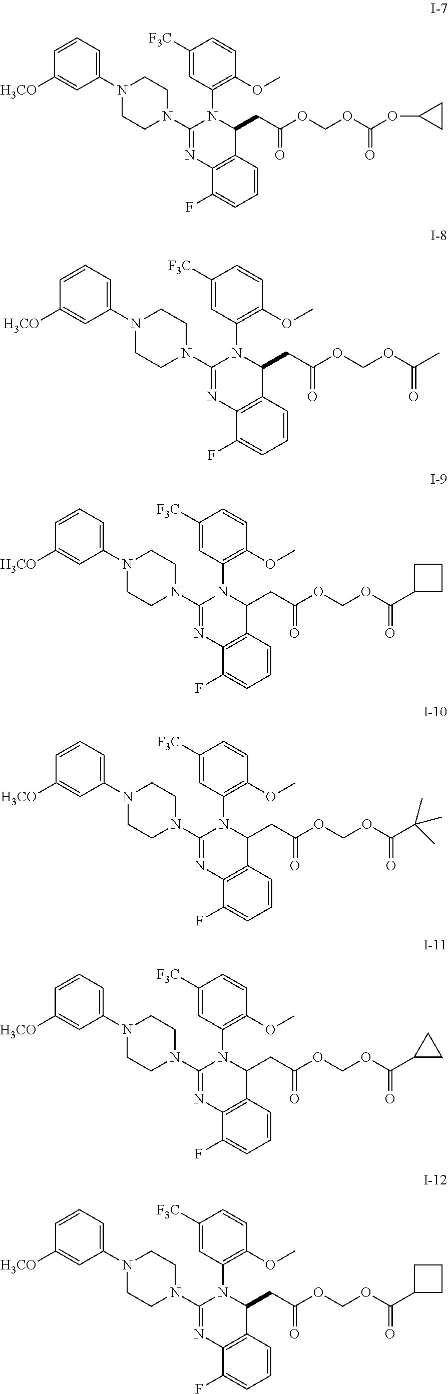

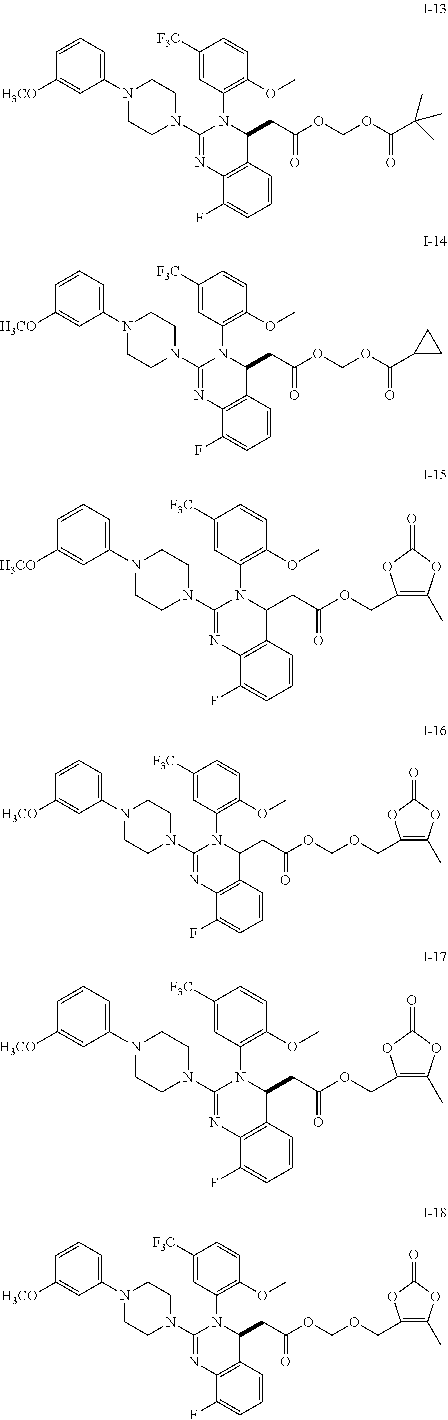

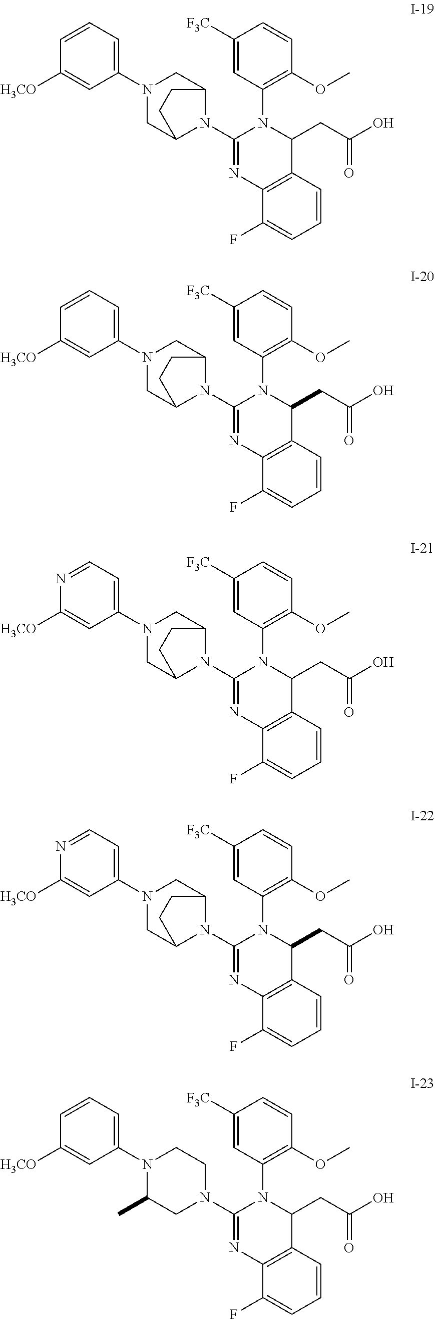 [SMILES: COc1cccc(N2CC3CCC(C2)N3C2=Nc3c(F)cccc3C(CC(=O)O)N2c2cc(C(F)(F)F)ccc2OC)c1 ; COc1cccc(N2CC3CCC(C2)N3C2=Nc3c(F)cccc3[C@H](CC(=O)O)N2c2cc(C(F)(F)F)ccc2OC)c1 ; COc1cc(N2CC3CCC(C2)N3C2=Nc3c(F)cccc3C(CC(=O)O)N2c2cc(C(F)(F)F)ccc2OC)ccn1 ; COc1cc(N2CC3CCC(C2)N3C2=Nc3c(F)cccc3[C@H](CC(=O)O)N2c2cc(C(F)(F)F)ccc2OC)ccn1 ; COc1cccc(N2CCN(C3=Nc4c(F)cccc4C(CC(=O)O)N3c3cc(C(F)(F)F)ccc3OC)C[C@H]2C)c1]

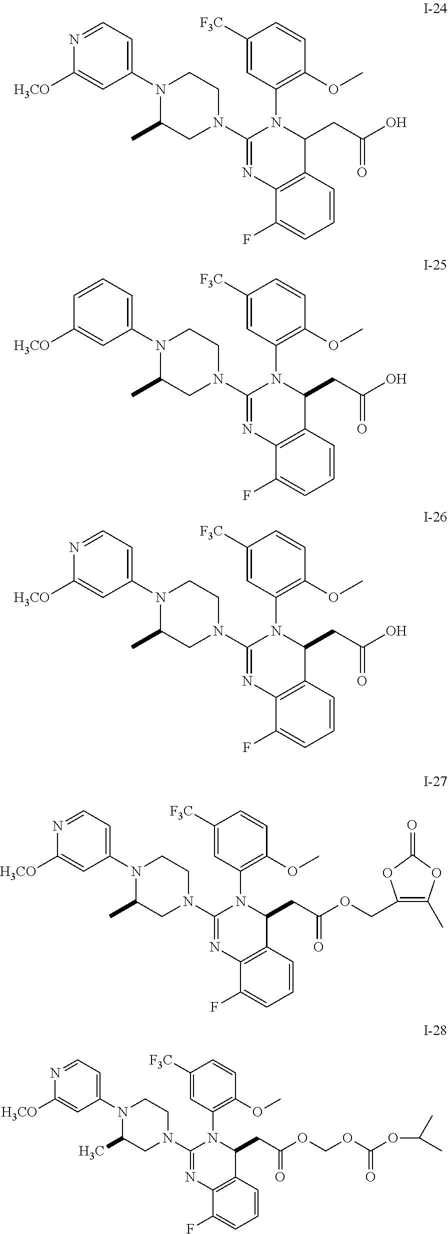

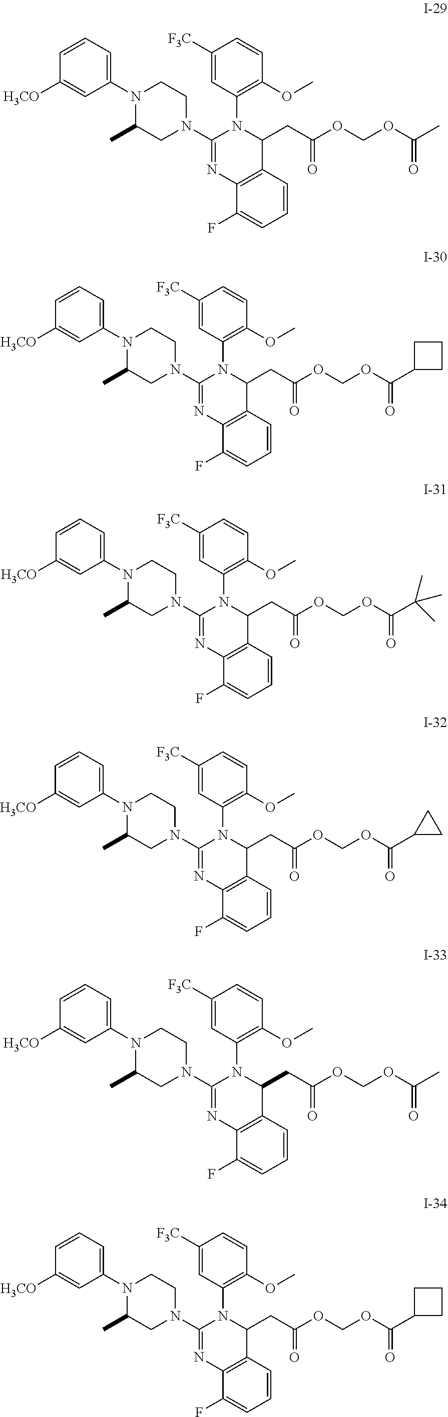

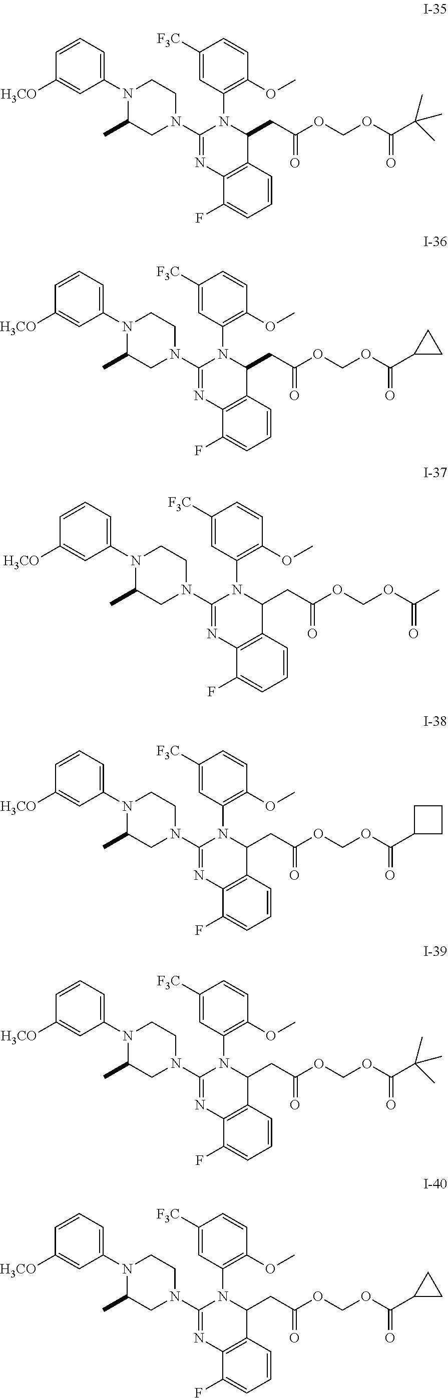

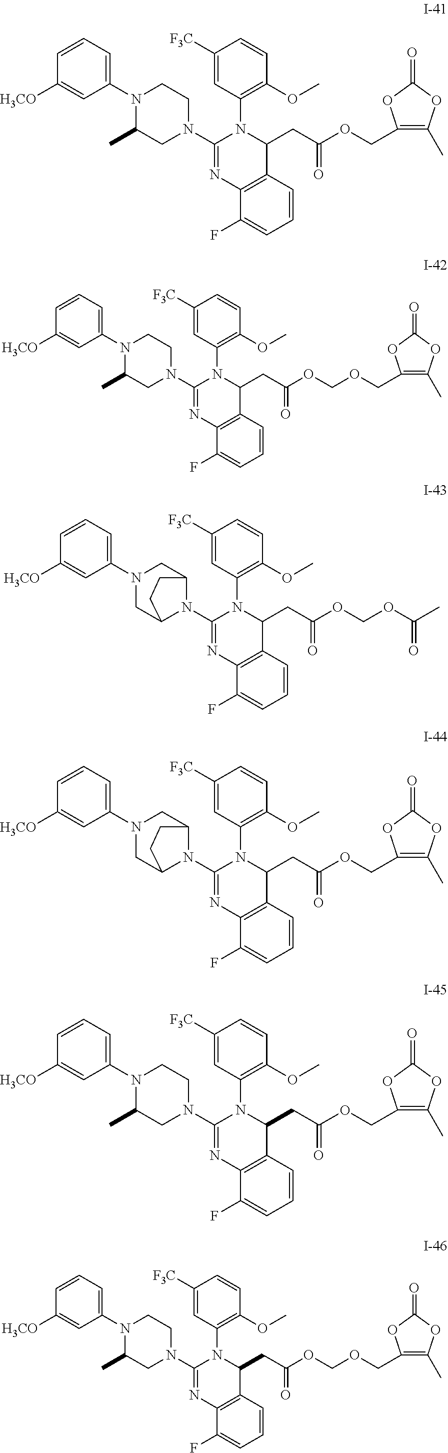

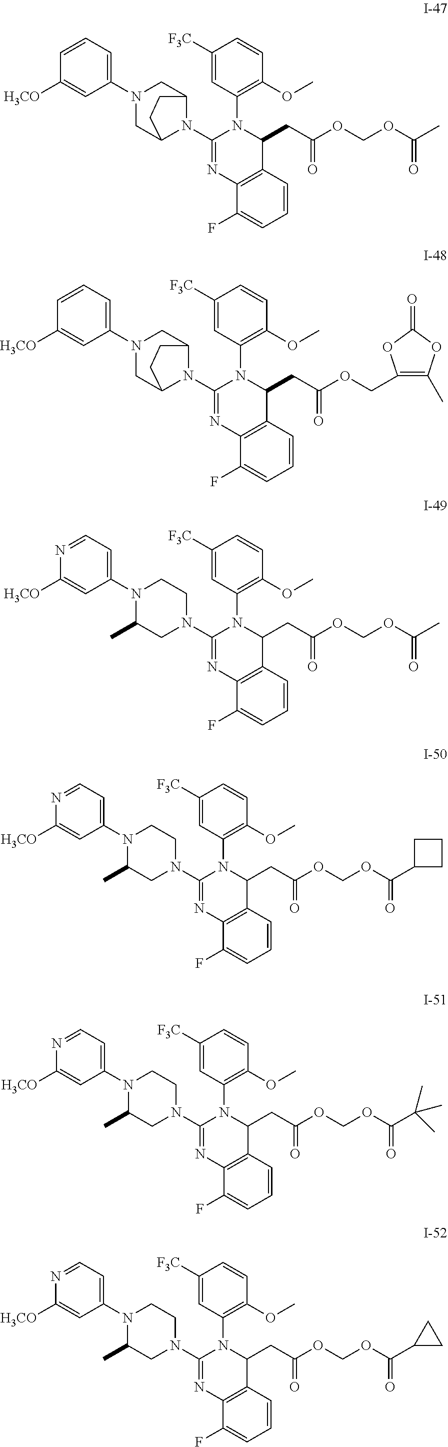

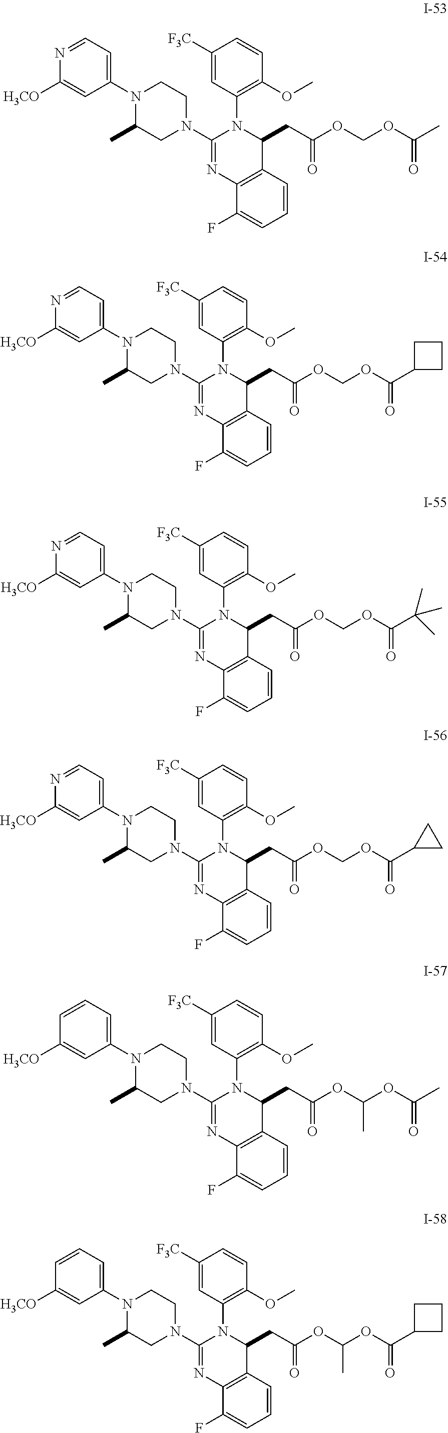

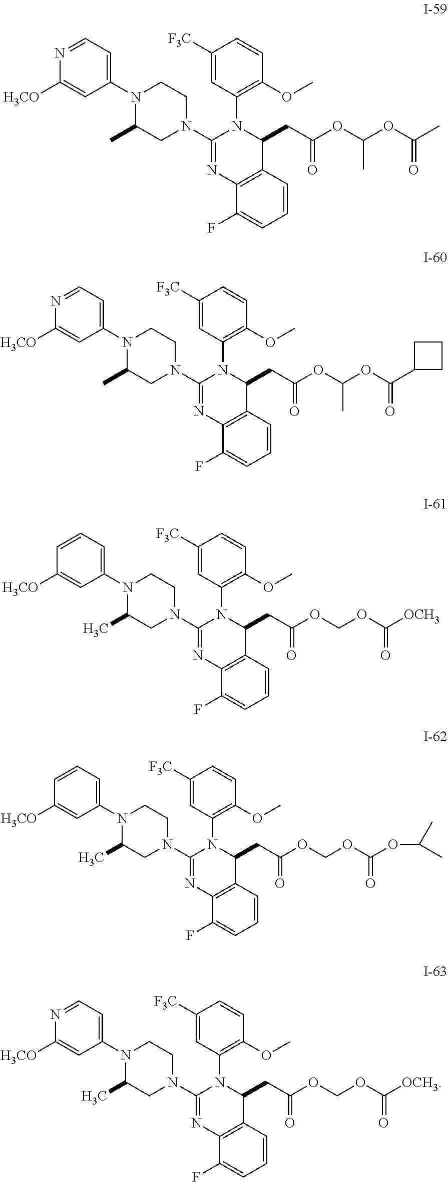

The inventor found by investigation that based on the low bioavailability of letermovir in HCT patients, and co-administration with cyclosporine to enhance the bioavailability in clinical practice, it is reasonable that letermovir is a substrate of efflux proteins, which is extensively expressed in gastrointestinal epithelial cells. For drugs, which are actively transported by protein as a carrier, usually have obvious polar or ionized groups. While the appropriate carboxyl esterification can reduce the polarity of drugs, ameliorate the absorption efficiency of drugs, mitigate the efflux effect of drugs, then increase the activity of drugs, and help to improve the bioavailability of drugs. Concurrently it is also possible that drugs do not require the help of cyclosporine in clinical practice, which can further reduce the toxicity of the drugs and improve patient compliance.

Furthermore, the present disclosure provides an R₁ substituent on the piperazine ring of letermovir, which can stabilize the bioactive conformation of this drug, significantly increase its potency. and provide the possibility for clinical use to avoid the combination with cyclosporine, and at the same time, highly active compounds may bring significant therapeutic or preventive antiviral outcomes. Due to the significant improvement of the activity of the compounds, the reduction in clinical dosage and improvement in toxicity, as well as strong suppression of viruses, this type of compounds could be more widely used in organ transplant patients.

The present disclosure also relates to a pharmaceutical composition comprising one or more heterocyclic derivatives or stereoisomers or pharmaceutically acceptable salts thereof according to the present disclosure, and a pharmaceutically acceptable carrier.

Preferably, the pharmaceutical composition is an anti-HCMV pharmaceutical composition.

In some embodiments, the pharmaceutical composition further comprises one or more therapeutic agents or preventive drugs, which are selected from the group consisting of vaccines, antibody drugs, antibody-drug conjugates (ADC drugs), nucleoside drugs, other drugs against human cytomegalovirus infection, and combinations thereof.

In some embodiments, the pharmaceutically acceptable carrier is selected from the group consisting of pharmaceutically acceptable diluents, excipients, fillers, adhesives, disintegrants, absorption enhancers, surfactants, lubricants, flavorings, sweeteners, and combinations thereof.

In some embodiments, the pharmaceutical composition is in a dosage form selected from tablets, powders, capsules, granules, oral liquids, injections, etc. The preferred dosage form for the pharmaceutical composition is tablets, capsules, or injections. The above-mentioned dosage form for the drug can be prepared using conventional methods in the pharmaceutical field.

In some embodiments, the pharmaceutically acceptable salt refers to a carboxylate salt of the compound of the present disclosure, which may be a sodium salt, potassium salt, calcium salt, ammonium salt, magnesium salt, or other similar metal salts.

In some embodiments, the pharmaceutically acceptable salt refers to a salt formed by addition with a pharmaceutically acceptable acid, which comprises inorganic and organic acids; the inorganic acids mentioned comprise hydrochloric acid, hydrobromic acid, sulfuric acid, phosphoric acid, bicarbonate, nitric acid, etc. The organic acids mentioned comprise acetic acid, ascorbic acid, benzenesulfonic acid, benzoic acid, hydroxyethyl sulfonic acid, ethanesulfonic acid, citric acid, edetic acid, fumaric acid, maleic acid, malic acid, tartaric acid, mandelic acid, gluconic acid, glutamic acid, lactic acid, dodecyl sulfonic acid, oxalic acid, stearic acid, succinic acid, p-toluenesulfonic acid, etc.

In some embodiments, the pharmaceutical composition comprises components in the following mass percentages:

| | |
|---|---|
| Heterocyclic derivative or a stereoisomer or | 5-95% |
| a pharmaceutically acceptable salt thereof | |
| Lactose | 1-60% |
| Starch | 0-20% |
| Microcrystalline cellulose | 1-40% |
| Sodium carboxymethyl starch | 1-5% |
| Polyethylene glycol | 0-10% |
| Magnesium stearate | 1-5%. |

Polyethylene glycol is preferably PEG6000.

The present disclosure also relates to a use of the aforementioned heterocyclic derivatives or stereoisomers or pharmaceutically acceptable salts thereof, and the aforementioned pharmaceutical compositions in the preparation of a medicine for the prevention and/or treatment of anti-human cytomegalovirus infection diseases.

When used alone or in the pharmaceutical composition, the compound according to the present disclosure is preferably present in a therapeutic/preventive effective amount.

The present disclosure further provides a compound suitable for preparing a heterocyclic derivative or a stereoisomer, a pharmaceutically acceptable salt thereof, the compound is represented by the following formula (II-c):

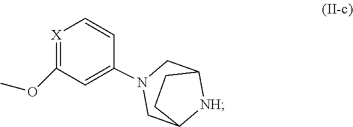

wherein:
X is selected from N and —CH—.

Typical Compounds (II-c) are, for example, the compounds synthesized in the following embodiments or compounds directly associated with them.

The present disclosure further provides a general scheme for preparing compounds of a heterocyclic derivative or a stereoisomer, a pharmaceutically acceptable salt thereof, which comprises the steps of reacting the aforementioned compound with the general formula (II-c) or

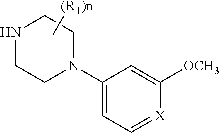

with a compound

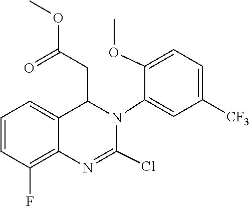 [SMILES: COC(=O)CC1c2cccc(F)c2N=C(Cl)N1c1cc(C(F)(F)F)ccc1OC]

to form a corresponding ester, hydrolyzing it to an acid, and finally further carrying out esterification to afford Compounds (I-c), (I-a), or (I-b).

In some embodiments, the general scheme comprises the following key steps:

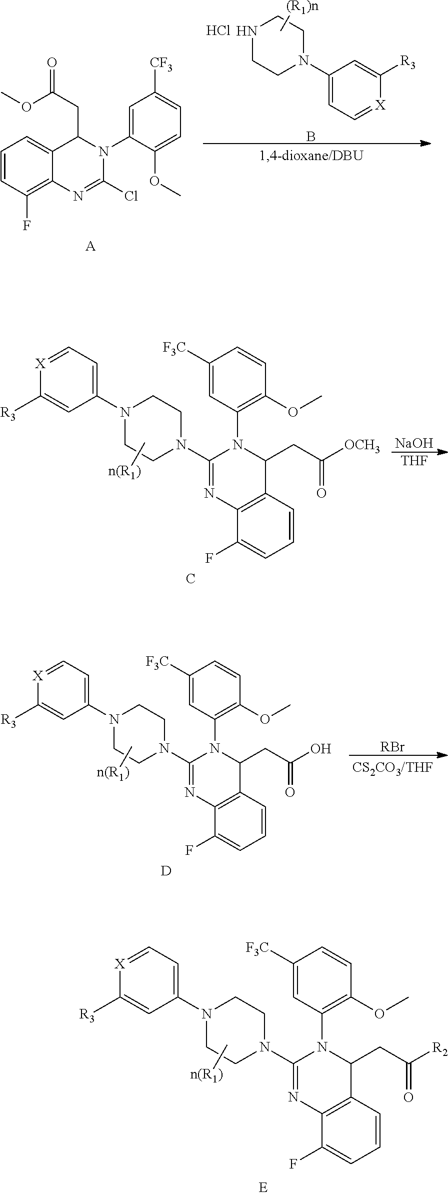

Step 1: 1,8-Diazabicyclo[5.4.0]undec-7-ene (DBU) was added to a solution of compound A and B in 1,4-dioxane, the reaction mixture was stirred at 80 to 100° C. for 1 to 12 h, then followed by workup and purification to give Intermediate C.

Step 2: Compound C was added to an aqueous solution of tetrahydrofuran (THF) and sodium hydroxide, then the reaction mixture was stirred at room temperature for 2 to 12 h. The reaction mixture was neutralized by dilute hydrochloric acid to pH 5-6, followed by concentration and purification to give Compound D.

Step 3: A prodrug moiety containing halogenated compound was added to an acetone solution of Compound D and cesium carbonate. The reaction mixture was heated at 40 to 100° C. for 1 to 12 h, cooled, diluted with water, extracted with ethyl acetate, then dried over anhydrous sodium sulfate, concentrated and purified to give Prodrug E.

Due to the embodiments of the above technical solutions, the present disclosure has the following advantages over the prior art:

By shielding the polarity of carboxyl groups and modulating the bioactive conformation of piperazine, the heterocyclic derivative of the present disclosure has higher potency in inhibiting HCMV, with an 8- to 40-fold increase in potency compared to letermovir, and may provide better protection for patients with HCMV infection after allogeneic hematopoietic stem cell transplantation, kidney transplantation, and lung transplantation in clinical practice.

### DETAILED DESCRIPTION OF EXEMPLARY EMBODIMENTS

### Definition of Terms

In the compounds described in the present disclosure, when any variable (for example, R₁, R₂, etc.) appears more than once in any component, the definition of each occurrence is independent of the definitions of other occurrences. Similarly, combinations of substituents and variables are allowed as long as such combinations stabilize the compound. The line drawn from a substituent into a ring system indicates that the bond referred to can be connected to any ring atom that can be substituted. In a broad sense, allowable substituents comprise substituents on acyclic and cyclic, branched and unbranched, carbocyclic and heterocyclic, aromatic and non-aromatic carbon and heteroatoms in organic compounds. It should be understood that those ordinary skilled in the art can choose the substituents and substitution forms of the compounds of the present disclosure to provide chemically stable compounds that can be easily synthesized from readily available raw materials through the techniques in the art and the methods proposed below. If a substituent itself is replaced by more than one functional group, it should be understood that these functional groups can be on the same carbon atom or different carbon atoms, as long as the structure is stabilized.

Unless otherwise defined, all technical and scientific terms used herein have the same meaning as commonly understood by those ordinary skilled in the art to which this disclosure belongs.

The term “stereoisomer” refers to an isomer produced by the different spatial arrangement of atoms in a molecule. It includes cis-trans-isomers, enantiomers and conformational isomers. All stereoisomers are within the scope of the present disclosure. The compounds of the present disclosure may be a single stereoisomer or a mixture of other isomers such as a racemate, or a mixture of all other stereoisomers.

The term “salt” refers to a pharmaceutically acceptable salt formed by a compound of the present disclosure with an acid, which may be an organic or inorganic acid, specifically selected from the group consisting of phosphoric acid, sulfuric acid, hydrochloric acid, hydrobromic acid, citric acid, maleic acid, malonic acid, mandelic acid, succinic acid, fumaric acid, acetic acid, lactic acid, nitric acid, sulfonic acid, p-toluenesulfonic acid, malic acid, methanesulfonic acid or analogues thereof.

The term “solvate” refers to a form of a compound of the present disclosure that forms a solid or liquid complex by coordination with a solvent molecule. Hydrates are a special form of solvates in which coordination occurs with water. Within the scope of the present disclosure, the solvate is preferably a hydrate.

The term “crystal” refers to the various solid forms formed by the compounds described herein, including crystalline forms and amorphous forms.

The term “hydrocarbyl” refers to saturated alkyl, alkenyl and alkynyl.

The term “hydrocarbyl” refers to a linear, branched or cyclic saturated or unsaturated substituent consisting of carbon and hydrogen. It has preferably 1 to 20 carbon atoms, more preferably 1 to 12 carbon atoms. The term “alkyl” refers to a linear, branched or cyclic saturated hydrocarbyl group. The alkyl group specifically includes methyl, ethyl, n-propyl, isopropyl, cyclopropyl, n-butyl, isobutyl, tert-butyl, cyclobutyl, n-pentyl, isopentyl, neopentyl, cyclohexyl, n-hexyl, isohexyl, 2,2,-dimethylbutyl and 2,3-dimethylbutyl, 16-alkyl, 18-alkyl. The term “C₁₋₂₀ alkyl” refers to a linear, branched or cyclic saturated hydrocarbyl group containing 1 to 20 carbon atoms. Alkyl includes substituted and unsubstituted alkyl. When an alkyl group is substituted, the substituent may substitute at any available point of attachment, and the substitution may be mono-substitution or poly-substitution. The substituent can be independently selected from the group consisting of alkyl, alkenyl, alkynyl, alkoxy, alkylthio, alkylamino, deuterum, halogen, thiol, hydroxyl, nitro, carboxy, ester, cyano, cycloalkyl, aryl, heteroaryl, cycloalkoxy, heterocycloalkoxy, cycloalkylthio, and oxo. The substituent is usually placed before alkyl when naming, for example, C₁₋₃ alkoxy C₃₋₈ cycloalkyl C₁₋₆ alkyl means C₁₋₆ alkyl is substituted by C₃₈ cycloalkyl, and C₃₋ₛ cycloalkyl is further substituted by C₁₋₃ alkoxy, for example: the structural formula of methoxycyclobutylmethyl is:

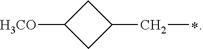

The term “alkenyl” and “alkynyl” refers to a linear, branched or cyclic unsaturated hydrocarbyl group containing a double bond and a triple bond, respectively, preferably containing 2 to 20 carbon atoms, more preferably 2 to 12 carbon atoms. Alkenyl and alkynyl include substituted and unsubstituted alkenyl and alkynyl, respectively. When they are substituted, the substituent may substitute at any available point of attachment, and the substitution may be mono-substitution or poly-substitution. The substituent can be independently selected from the group consisting of alkyl, alkenyl, alkynyl, alkoxy, alkylthio, alkylamino, deuterum, halogen, thiol, hydroxyl, nitro, carboxy, ester, cyano, cycloalkyl, aryl, heteroaryl, cycloalkoxy, heterocycloalkoxy, cycloalkylthio, and oxo. The substituent is usually placed before alkenyl and alkynyl when naming.

The term “ring” refers to carbocyclic and heterocyclic rings. The term “carbocyclyl” or “carbocyclic ring” refers to carbocyclyl having 3 to 20 carbon atoms, preferably 3 to 16 carbon atoms, more preferably 4 to 12 carbon atoms, and includes cycloalkyl, cycloalkenyl, aryl, bicyclic carbocyclyl, polycyclic carbocyclyl, and the like. “Heterocyclyl” or “heterocyclic ring” contains heteroaryl, non-aromatic heterocyclyl, bicyclic heterocyclyl, polycyclic heterocyclyl and the like containing in the ring one or more identical or different heteroatoms selected from O, S and N. The term “ring” includes monocyclic rings, bridged rings, spiro rings, fused rings, and polycyclic rings.

The term “cyclohydrocarbyl” refers to saturated and/or partially unsaturated monocyclic or polycyclic cyclohydrocarbyl. A monocyclic ring may include 3 to 10 carbon atoms. Non-limiting examples of monocyclic cyclohydrocarbyl hydrocarbyl include cyclopropyl, cyclobutyl, cyclopentyl, cyclopentenyl, cyclohexyl, cyclohexenyl, cyclohexadienyl, cycloheptyl and the like. Ploycyclic cycloalkyl includes spiro cycloalkyl, fused cycloalkyl, and bridged cycloalkyl. Cycloalkyl includes non-substituted and substituted ones. The substituent comprises one or more substituents independently selected from the following groups including but not limited to, alkyl, cycloalkyl, alkoxy, halogen, carboxyl, ester, amino, acylamino, hydroxyl, cyano, nitro, aryl, heteroaryl.

The term “aryl” refers to carbocyclic aryl and heteroaryl.

The term “carbocyclic aryl” refers to a 6- to 10-membered all-carbon monocyclic or polycyclic aromatic group, including phenyl, naphthyl, biphenyl, and the like. Aryl can be substituted and unsubstituted. The substituent is independently selected from the group consisting of alkyl, cycloalkyl (cyclopropyl, cyclobutyl, cyclopentyl, etc.), alkenyl, alkynyl, azide, amino, deuterium, alkoxy, alkylthio, alkylamino, halogen, thiol, hydroxyl, nitro, heterocycloalkyl, aryl, heteroaryl, cycloalkoxy, heterocycloalkoxy, cycloalkylthio, heterocycloalkylthio, alkylsilyl and so on.

The term “heteroaryl” refers to a group of a heteroaromatic system containing 1 to 10 heteroatoms. Heteroatoms include oxygen, sulfur, nitrogen, phosphorus, and the like. Wherein monoheterocyclyl includes, but is not limited to, furan, thiophene, pyrrole, thiazole, imidazole, 1,2,3-triazole, 1,2,4-triazole, 1,2,3-thiadiazole, oxazole, 1,2,4-oxadiazole, 1,3,4-oxadiazole, pyridine, pyrimidine, pyridazine, pyrazine, tetrahydrofuran, tetrahydropyrrole, piperidine, piperazine, morpholine, isoxazolin and the like. Fused heterocyclyl includes, but is not limited to, quinoline, isoquinoline, indole, benzofuran, benzothiophene, purine, acridine, carbazole, fluorene, chromenone, fluorenone, quinoxaline, 3, 4-dihydronaphthalen, dibenzofuran, hydrogenated dibenzofuran, benzoxazolyl, and the like. Heteroaryl can be substituted and unsubstituted. The substituent is independently selected from the group consisting of alkyl, cycloalkyl (cyclopropyl, cyclobutyl, cyclopentyl, etc.), alkenyl, alkynyl, azide, amino, deuterium, alkoxy, alkylthio, alkylamino, halogen, thiol, hydroxyl, nitro, heterocycloalkyl, aryl, heteroaryl, cycloalkoxy, heterocycloalkoxy, cycloalkylthio, heterocycloalkylthio, alkylsilyl and so on.

The term “halogen” refers to fluorine, chlorine, bromine and iodine, preferably fluorine, chlorine and bromine.

The term “deuterium” is an isotope of hydrogen, the atomic mass is twice that of the latter, and the binding to carbon is stronger. “Deuterated” and “deuterium” means that hydrogen is replaced with deuterium at the specified position. A “deuterated substituent” is a substituent in which at least one hydrogen is replaced with deuterium enriched at a specified percentage.

The term “haloalkyl” refers to alkyl substituted with at least one halogen atom.

The term “heterocyclyl” means a cyclic group containing at least one hetero atom, wherein the hetero atom is nitrogen, oxygen, sulfur, and the like. Heterocyclyl includes monoheterocyclyl and polyheterocyclyl.

In addition to the standard processes known in the literature or demonstrated in experimental procedures, the following synthetic schemes can be used to prepare the compounds of the present disclosure.

By combining the following synthesis schemes, it can better understand the compounds and synthesis methods described in the present disclosure. The described synthesis schemes describe a process that can be used to prepare the compounds described in the present disclosure, and the described process is only an illustrative scheme description for illustrative purposes and does not constitute a limitation on the scope of the present disclosure.

The present disclosure will be further described in combination with embodiments, but these embodiments are not intended to limit the scope of protection of the present disclosure.

The various technical features of the embodiments described below can be combined in any way, in order to make the description concise, not all possible combinations of the various technical features in the embodiments described below have been described, however, as long as there is no contradiction in the combination of these technical features, they should be considered within the scope of this specification.

The embodiments described below only give expression to several implementations of the present disclosure, and their descriptions are more specific and detailed, but should not be understood as limiting the scope of the disclosure patent. It should be pointed out that for those ordinary skilled in the art, several modifications and improvements can be made without departing from the inventive concept, which are within the scope of protection of the present disclosure. Therefore, the scope of protection of this disclosure patent should be based on the appended claims.

The abbreviations of the compound names used in the embodiments are as follows:
DBU: 1,8-diazabicyclo[5.4.0]undec-7-ene
DPPA: Diphenyl phosphoryl azide
DCM: Dichloromethane
EtOAc: Ethyl acetate
PE: Petroleum ether
EA: Ethyl acetate
THF: Tetrahydrofuran
MTBE: Methyl tert-butyl ether
1,4-Dioxane: 1,4-dioxane
TFA: Trifluoroacetic acid
c-Hex₂NMe: N,N-Dicyclohexylmethylamine
Pd₂(dba)₃: Tris(dibenzylideneacetone)dipalladium
X-Phos: dicyclohexyl-[2-[2,4,6-tri(propan-2-yl)phenyl]phenyl]phosphane.

### Example 1: Synthesis of Compound I-5

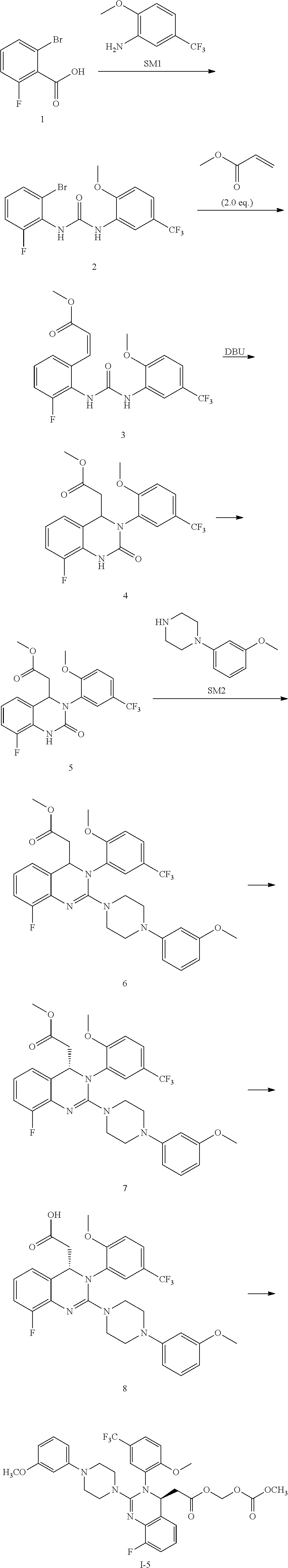

Synthesis of Compound 2: A weighted Compound 1 (10.00 g, 45.66 mmol) was dissolved in toluene (100 ml) and cooled under ice bath, DPPA (18.85 g, 68.49 mmol) and triethylamine (13.86 g, 136.98 mmol) were added to the reaction mixture and stirred at room temperature for 1 h. The reaction mixture was heated to 80° C. and stirred for 1 h, SM1

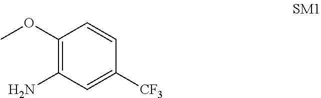

(12.22 g, 63.92 mmol) was added, and continued stirring for 2 h. The reaction solution was cooled and poured into water (300 ml), then extracted twice with ethyl acetate (300 ml), the organic layers were washed with saturated saline solution (300 ml), dried over anhydrous sodium sulfate, filtered, and concentrated under reduced pressure to give a crude product, which was purified by pulping (PE:EA=3:1, 200 ml) to give 15.9 g of gray solid. Synthesis of Compound 3: To a solution of Compound 2 (11.40 g, 27.99 mmol) in isopropyl acetate (80 mL) was added methyl acrylate (7.23 g, 83.97 mmol), c-Hex₂NMe (6.56 g, 33.59 mmol) and (t-Bu)₃P—Pd (1.43 g, 2.80 mmol), then heated to 80° C. and stirred for 4 h under nitrogen atmosphere. The reaction solution was cooled and poured into water (300 ml), then extracted twice with ethyl acetate (300 ml), the organic layers were passed through a silica gel pad, the silica gel pad was washed with ethyl acetate (300 ml), and concentrated under reduced pressure to give a crude product, which was purified by pulping (PE:EA=5:1, 150 ml) to give 8.4 g of white solid.

Synthesis of Compound 4: Compound 3 (7.7 g, 18.67 mmol) and DBU (1.42 g, 9.34 mmol) were dissolved in toluene (120 ml), and the reaction mixture was heated to 100° C. and stirred for 1 h. The reaction solution was cooled and poured into water (200 ml), then extracted twice with DCM (200 ml), the organic layers were dried over anhydrous sodium sulfate, filtered, and concentrated under reduced pressure to give a crude product, which was purified by pulping (PE:EA=5:1, 50 ml) to give 6.4 g of yellow solid.

Synthesis of Compound 5: Compound 4 (6.4 g, 15.19 mmol) was added into phosphorus oxychloride (100 ml), and the reaction mixture was heated to 105° C. and stirred for 3 h. The reaction solution was concentrated under reduced pressure to remove most of phosphorus oxychloride, cooled and poured into MTBE (300 ml), and layered with water (300 ml), then the organic layer was washed with saturated sodium bicarbonate (300 ml), dried over anhydrous sodium sulfate, filtered, and concentrated under reduced pressure to give a crude product, which was purified by column chromatography (PE:EA=10:1) to give 6.6 g of yellow oily substance.

Synthesis of Compound 6: A weighted Compound 5 (6.6 g, 15.32 mmol) was dissolved in 1,4-dioxane (60 ml), SM2

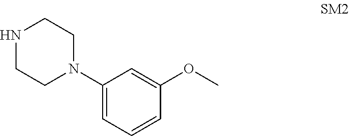

(3.5 g, 15.32 mmol) and DBU (7.0 g, 45.96 mmol) were added. The reaction mixture was heated to 100° C., stirred for 1 h, then the reaction mixture was cooled and poured into water (200 ml), extracted with ethyl acetate (200 ml), the organic layer was dried over anhydrous sodium sulfate, filtered, and concentrated under reduced pressure to give a crude product, which was purified by column chromatography (PE:EA=10:1 ˜DCM:MeOH=100:1, during the column chromatography purification, the elution phase PE and EA (v/v was 10:1) was used first, followed by DCM and MeOH (v/v was 100:1)) to give 6.5 g of light yellow solid.

Synthesis of Compound 7: Compound 6 (6.5 g, 11.08 mmol) was chirally separated by preparative liquid chromatography to give 2.8 g of off-white solid.

Synthesis of Compound 8: Into a solution of a weighted Compound 7 (2.00 g, 3.41 mmol) in THF (27 ml) was added 0.5M sodium hydroxide aqueous solution (27 ml, 13.64 mmol), then the reaction mixture was stirred at room temperature for 4 h. The reaction solution was poured into water (200 ml), neutralized with citric acid solid to pH 5˜6, and extracted with MTBE (200 ml), the organic layer was dried over anhydrous sodium sulfate, filtered, and concentrated under reduced pressure to give 1.8 g of white solid.

Synthesis of Compound I-5: To a solution of a weighted Compound 8 (200 mg, 0.35 mmol) in acetone (5 ml) was added chloromethyl dimethyl carbonate (66 mg, 0.53 mmol), potassium iodide (58 mg, 0.35 mmol) and cesium carbonate (456 mg, 1.40 mmol), then the reaction mixture was heated to 56° C. and stirred for 2 h. The reaction solution was poured into water (100 ml), and extracted with ethyl acetate (100 ml), the organic layer was washed with saturated saline solution (100 ml), dried over anhydrous sodium sulfate, filtered, and concentrated under reduced pressure to give a crude product, which was purified by column chromatography preparation plate (PE:EA=2:1) to give 120 mg of white solid. LCMS ESI m/z: 661.6 (M+H)⁺; ¹H NMR (400 MHz, Chloroform-d): δ 7.40 (d, J=8.0 Hz, 1H), 7.16-7.12 (m, 2H), 7.04-6.99 (m, 2H), 6.90-6.85 (m, 1H), 6.76 (d, J=4.0 Hz, 1H), 6.46-6.40 (m, 2H), 6.37 (s, 1H), 5.84 (d, J=4.0 Hz, 1H), 5.70 (d, J=4.0 Hz, 1H), 4.89-4.85 (m, 1H), 3.90 (s, 3H), 3.81 (s, 3H), 3.76 (s, 3H), 3.53 (d, J=36.0 Hz, 4H), 3.07-2.87 (m, 5H), 2.71-2.66 (m, 1H).

### Example 2: Synthesis of Compound I-8

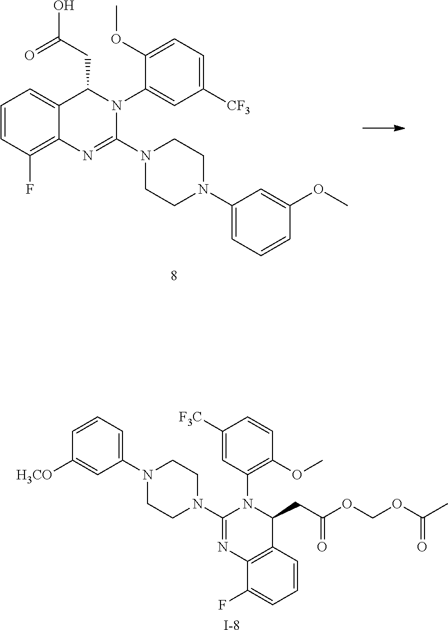

To a solution of a weighted Compound 8 (250 mg, 0.44 mmol) in acetone (5 ml) was added bromomethyl acetate (101 mg, 0.66 mmol) and cesium carbonate (573 mg, 1.76 mmol), then the reaction mixture was heated to 56° C. and stirred for 2 h. The reaction solution was poured into water (100 ml), and extracted with ethyl acetate (100 ml), the organic layer was washed with saturated saline solution (100 ml), dried over anhydrous sodium sulfate, filtered, and concentrated under reduced pressure to give a crude product, which was purified by column chromatography preparation plate (PE:EA=2:1) to give 88 mg of white solid. LCMS ESI m/z: 645.6 (M+H)⁺; ¹H NMR (400 MHz, Chloroform-d): δ 7.40 (d, J=8.4 Hz, 1H), 7.16-7.12 (m, 2H), 7.04-6.99 (m, 2H), 6.90-6.85 (m, 1H), 6.77-6.76 (m, 1H), 6.46-6.37 (m, 3H), 5.80-5.69 (m, 2H), 4.87-4.84 (m, 1H), 3.90 (s, 3H), 3.76 (s, 3H), 3.53 (d, J=36.0 Hz, 4H), 3.09-2.79 (m, 5H), 2.72-2.67 (m, 1H), 2.08 (s, 3H).

### Example 3: Synthesis of Compound I-13

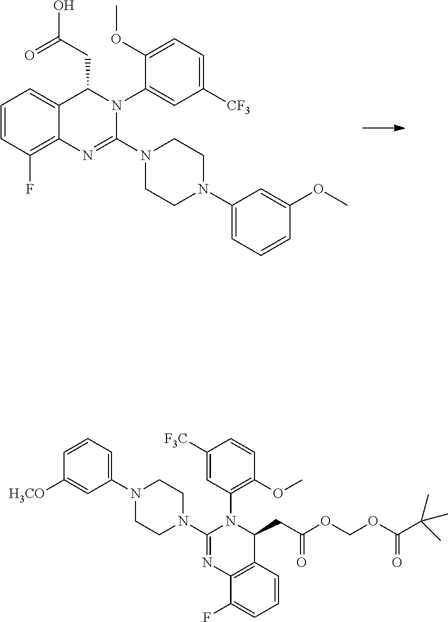 [SMILES: COc1cccc(N2CCN(C3=Nc4c(F)cccc4[C@H](CC(=O)O)N3c3cc(C(F)(F)F)ccc3OC)CC2)c1 ; COc1cccc(N2CCN(C3=Nc4c(F)cccc4[C@H](CC(=O)OCOC(=O)C(C)(C)C)N3c3cc(C(F)(F)F)ccc3OC)CC2)c1]

To a solution of a weighted Compound 8 (250 mg, 0.44 mmol) in acetone (5 ml) was added chloromethyl pivalate (99 mg, 0.66 mmol), potassium iodide (73 mg, 0.44 mmol) and cesium carbonate (573 mg, 1.76 mmol), then the reaction mixture was heated to 56° C. and stirred for 2 h. The reaction solution was poured into water (100 ml), and extracted with ethyl acetate (100 ml), the organic layer was washed with saturated saline solution (100 ml), dried over anhydrous sodium sulfate, filtered, and concentrated under reduced pressure to give a crude product, which was purified by column chromatography preparation plate (PE:EA=2:1) to give 130 mg of white solid. LCMS ESI m/z: 687.7 (M+H)⁺; ¹H NMR (400 MHz, Chloroform-d): δ 7.40 (d, J=12.0 Hz, 1H), 7.16-7.11 (m, 2H), 7.04-6.99 (m, 2H), 6.89-6.84 (m, 1H), 6.79-6.77 (m, 1H), 6.46-6.36 (m, 3H), 5.78 (d, J=8.0 Hz, 1H), 5.72 (d, J=8.0 Hz, 1H), 4.87-4.84 (m, 1H), 3.91 (s, 3H), 3.76 (s, 3H), 3.53 (d, J=36.0 Hz, 4H), 3.09-2.79 (m, 5H), 2.74-2.68 (m, 1H), 1.18 (s, 9H).

### Example 4: Synthesis of Compound I-17

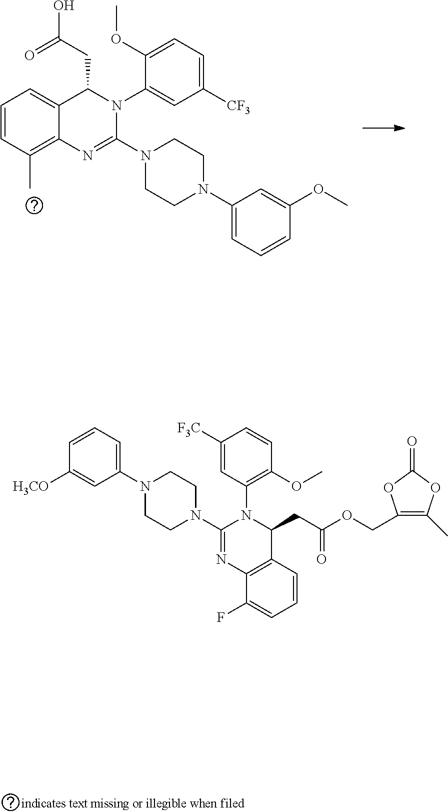 [SMILES: COc1cccc(N2CCN(C3=Nc4c(C)cccc4[C@H](CC(=O)O)N3c3cc(C(F)(F)F)ccc3OC)CC2)c1 ; COc1cccc(N2CCN(C3=Nc4c(F)cccc4[C@H](CC(=O)OCc4oc(=O)oc4C)N3c3cc(C(F)(F)F)ccc3OC)CC2)c1]

To a solution of a weighted Compound 8 (200 mg, 0.35 mmol) in acetone (5 ml) was added 4-chloromethyl-5-methyl-1,3-dioxol-2-one (79 mg, 0.53 mmol), potassium iodide (58 mg, 0.35 mmol) and cesium carbonate (456 mg, 1.40 mmol), then the reaction mixture was heated to 56° C. and stirred for 2 h. The reaction solution was poured into water (100 ml), and extracted with ethyl acetate (100 ml), the organic layer was washed with saturated saline solution (100 ml), dried over anhydrous sodium sulfate, filtered, and concentrated under reduced pressure to give a crude product, which was purified by column chromatography preparation plate (PE:EA=2:1) to give 125 mg of white solid. LCMS ESI m/z: 685.6 (M+H)⁺; ¹H NMR (400 MHz, Chloroform-d): δ 7.41 (d, J=8.0 Hz, 1H), 7.16-7.12 (m, 2H), 7.04-7.00 (m, 2H), 6.89-6.84 (m, 1H), 6.70 (d, J=8.0 Hz, 1H), 6.46-6.40 (m, 2H), 6.37 (s, 1H), 4.89-4.82 (m, 2H), 3.91 (s, 3H), 3.76 (s, 3H), 3.53 (d, J=36.0 Hz, 4H), 3.04-2.79 (m, 5H), 2.75-2.69 (m, 1H), 2.15 (s, 3H).

### Example 5: Synthesis of Compound I-19

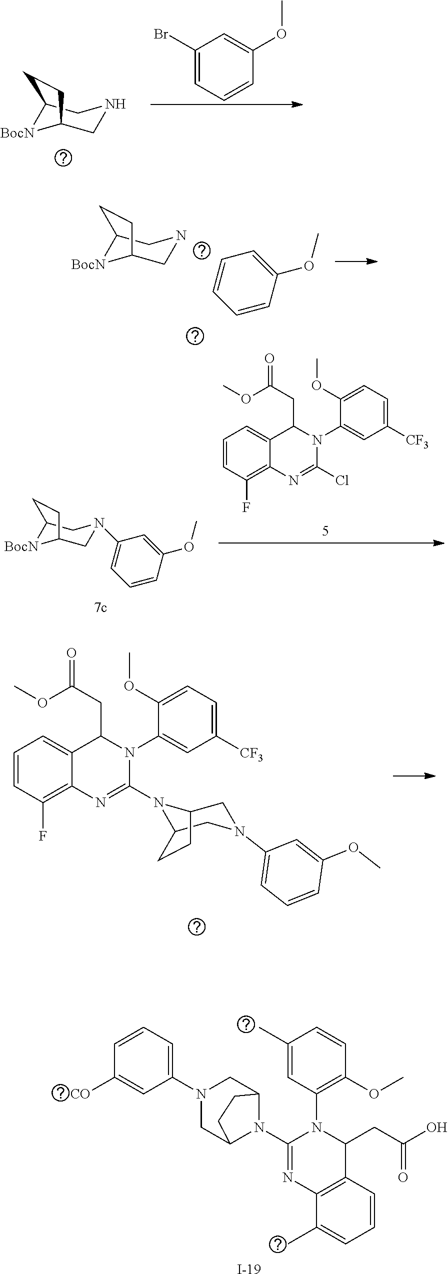

Synthesis of Compound 7b: Compound 7a (500 mg, 2.36 mmol), 3-bromoanisole (662 mg, 3.54 mmol), Pd₂(dba)₃ (18 mg, 0.02 mmol), tri-tert-butylphosphine (40 mg, 0.02 mmol) and potassium tert-butoxide (397 mg, 3.54 mmol) were weighted and dissolved in toluene (10 ml). The reaction mixture was heated to 100° C., stirred for 6 h under nitrogen atmosphere. The reaction mixture was cooled, poured into water (200 ml), and extracted with ethyl acetate (200 ml), the organic layer was washed with brine (200 ml), dried over anhydrous sodium sulfate, filtered, and concentrated under reduced pressure to give a crude product, which was purified by column chromatography preparation plate (PE:EA=10:1) to give 620 mg of yellow solid.

Synthesis of Compound 7c: To A solution of Compound 7b (620 mg, 1.95 mmol) in DCM (6 ml) was added TFA (3 ml) dropwise, then the mixture was stirred at room temperature for 1 h. The reaction solution was poured into water (100 ml), adjusted to pH 8˜9 with saturated sodium bicarbonate aqueous solution (100 ml), and extracted with ethyl acetate (100 ml), the organic layer was washed with brine (100 ml), dried over anhydrous sodium sulfate, filtered, and concentrated under reduced pressure to give a crude product, 280 mg of yellow solid.

Synthesis of Compound 7d: Compound 5 (180 mg, 0.42 mmol), Compound 7c (92 mg, 0.42 mmol), potassium iodide (6.6 mg, 0.04 mmol), and potassium carbonate (174 mg, 1.26 mmol) were weighted and dissolved in NMP (3 ml). The reaction mixture was heated to 80° C. and stirred for 1 h. The reaction mixture was cooled, poured into water (100 ml), then extracted with ethyl acetate (100 ml), the organic layer was washed with brine (100 ml), dried over anhydrous sodium sulfate, filtered, and concentrated under reduced pressure to give a crude product, which was purified by column chromatography preparation plate (PE:EA=5:1) to give 55 mg of yellow solid.

Synthesis of Compound I-19: To a solution of a weighted Compound 7d (55 mg, 0.09 mmol) in THF (1 ml) was added 0.5M sodium hydroxide aqueous solution (0.7 ml, 0.44 mmol), then the reaction mixture was stirred at room temperature for 4 h. The reaction mixture was poured into water (100 ml), neutralized with citric acid solid to pH to 5˜6, then extracted with ethyl acetate (100 ml), the organic layer was dried over anhydrous sodium sulfate, filtered, and concentrated under reduced pressure to give a crude product, which was purified by column chromatography (DCM:MeOH=20:1) to give 14 mg of white solid. LCMS ESI m/z: 599.6 (M+H)⁺; ¹H NMR (400 MHz, Chloroform-d): δ 7.40 (d, J=8.0 Hz, 1H), 7.11-6.92 (m, 4H), 6.80-6.70 (m, 2H), 6.34-6.22 (m, 3H), 4.83 (s, 1H), 4.50-4.27 (m, 3H), 3.83 (s, 3H), 3.74 (s, 3H), 3.66 (s, 1H), 3.34 (d, J=4.0 Hz, 1H), 3.14 (d, J=4.0 Hz, 1H), 2.98-2.94 (m, 1H), 2.72 (d, J=8.0 Hz, 1H), 2.65-2.60 (m, 1H), 1.70 (d, J=40.0 Hz, 4H).

### Example 6: Synthesis of Compounds I-23 and I-29

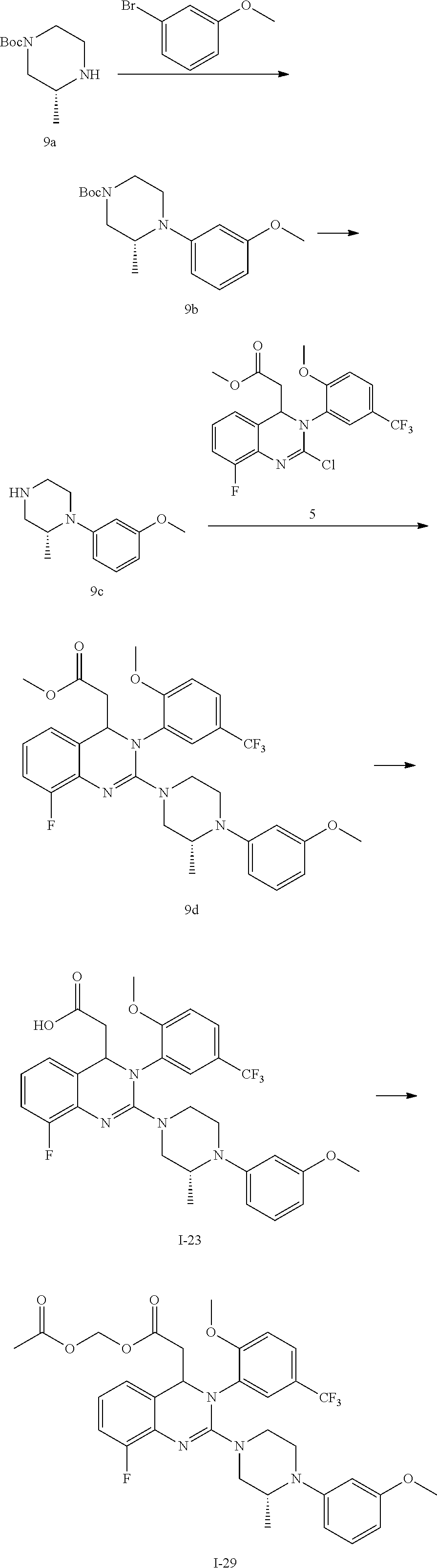

Synthesis of Compound 9b: Compound 9a (1.00 g, 5.00 mmol), 3-bromoanisole (1.12 g, 6.00 mmol), X-Phos (95 mg, 0.20 mmol), Pd₂(dba)₃ (183 mg, 0.20 mmol), and cesium carbonate (3.16 g, 10.00 mmol) were dissolved in 1,4-dioxane (10 ml), and the reaction mixture was heated to 100° C. and stirred for 3 h under nitrogen atmosphere. The reaction solution was cooled, poured into water (10 ml), and extracted with ethyl acetate (10 ml), the organic layer was washed with brine (10 ml), dried over anhydrous sodium sulfate, filtered, and concentrated under reduced pressure to give a crude product, which was purified by column chromatography (PE:EA=8:1) to give 1.3 g of yellow oily compound.

Synthesis of Compound 9c: To a solution of Compound 9b (1.3 g, 4.24 mmol) in DCM (10 ml) was added TFA (5 ml), then the reaction mixture was stirred at room temperature for 2 h. The reaction solution was diluted with ethyl acetate (200 ml), then neutralized by saturated sodium bicarbonate aqueous solution (200 ml) to pH 7˜8; the organic layer was dried over anhydrous sodium sulfate, filtered, and concentrated under reduced pressure to give a crude product, which was purified by pulping (PE:EA=5:1, 20 ml) to give 1.04 g of pink solid.

Synthesis of Compound 9d: Compound 9c (239 mg, 1.16 mmol) and DBU (457 mg, 3.48 mmol) were added to a solution of a weighted Compound 5 (500 mg, 1.16 mmol) in 1,4-dioxane (5 ml), then the reaction mixture was heated to 100° C. and stirred for 1 h. The reaction solution was cooled, poured into water (200 ml), and extracted with ethyl acetate (200 ml), the organic layer was washed with brine (200 ml), dried over anhydrous sodium sulfate, filtered, and concentrated under reduced pressure to give a crude product, which was purified by column chromatography preparation plate (PE:EA=5:1) to give 137 mg of white solid.

Synthesis of Compound I-23: To a solution of Compound 9d (137 mg, 0.23 mmol) in THF (2 ml) was added 0.5M sodium hydroxide aqueous solution (1.8 ml, 0.92 mmol), then the reaction mixture was stirred at room temperature for 2 h. The reaction solution was adjusted to pH 5˜6 with dilute hydrochloric acid, concentrated under reduced pressure to remove THF and water to give a crude product, the crude product was dissolved with DCM:MeOH=10: 1 (50 ml), and filtered to remove the insoluble matter, the filtrate was concentrated and purified by column chromatography to give 73 mg of white solid. LCMS ESI m/z: 587.3 (M+H)⁺; ¹H NMR (400 MHz, Chloroform-d): δ 7.48-7.38 (m, 1H), 7.13-7.09 (m, 1H), 7.03-6.86 (m, 3H), 6.82-6.80 (m, 1H), 6.46-6.22 (m, 3H), 4.88-4.83 (m, 1H), 3.83 (s, 2H), 3.74 (d, J=1.6 Hz, 3H), 3.73 (s, 6H), 3.55 (s, 1H), 3.38 (s, 1H), 3.07-2.91 (m, 2H), 2.68 (s, 1H), 2.02 (d, J=4.0 Hz, 2H).

Synthesis of Compound I-29: To a solution of Compound I-25 (61 mg, 0.10 mmol) in acetone (2 ml) was added cesium carbonate (98 mg, 0.30 mmol) and bromomethyl acetate (23 mg, 0.15 mmol), then the reaction mixture was heated to 56° C. and stirred for 1 h. The reaction solution was poured into water (50 ml), and extracted twice with ethyl acetate (50 ml), the organic layers were dried over anhydrous sodium sulfate, filtered, and concentrated under reduced pressure to give a crude product, which was purified by column chromatography preparation plate (DCM:MeOH=20:1) to give 15 mg of white solid. LCMS ESI m/z: 659.4 (M+H)⁺; ¹H NMR (400 MHz, Chloroform-d): δ 7.45-7.38 (m, 1H), 7.13 (t, J=8.0 Hz, 1H), 7.06-6.97 (m, 2H), 6.95-6.82 (m, 1H), 6.77 (d, J=4.0 Hz, 1H), 6.45-6.41 (m, 1H), 6.41-6.32 (m, 2H), 5.80-5.68 (m, 1H), 5.36-5.35 (m, 1H), 4.87-4.79 (m, 1H), 3.92 (s, 3H), 3.84-3.53 (m, 6H), 3.53-3.24 (m, 2H), 3.17-2.85 (m, 3H), 2.79-2.63 (m, 1H), 2.09 (t, J=4.0 Hz, 3H), 2.07-2.06 (m, 1H).

### Example 7: Synthesis of Compounds I-24 and I-49

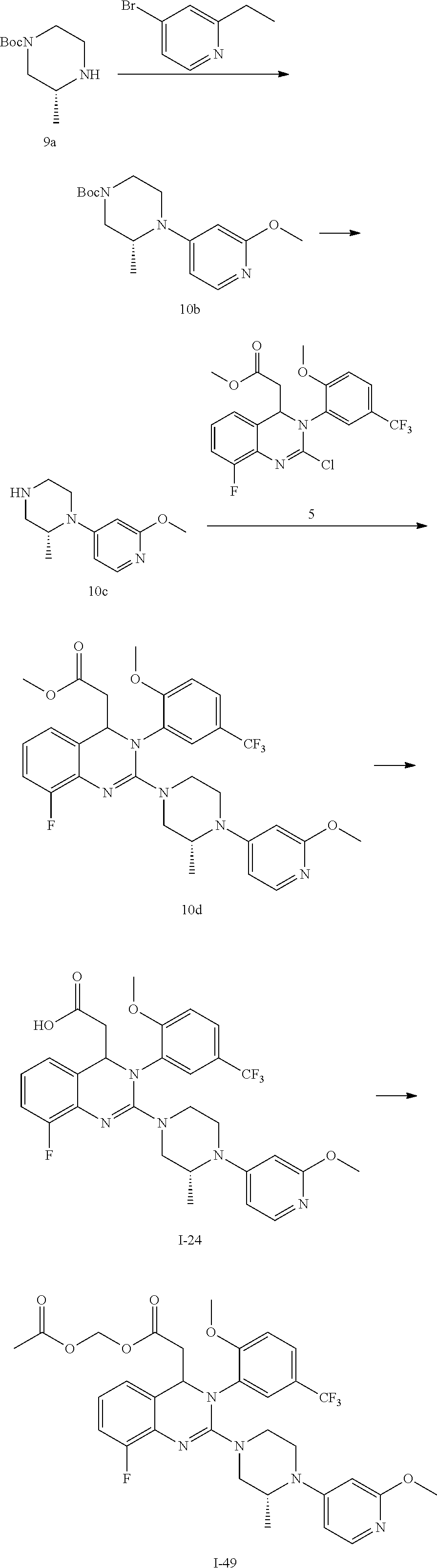

Synthesis of Compound 10b: Compound 9a (100 gg 5500 mmol), 4-bromo-2-methoxypyridine (1.13 g, 6.00 mmol), X-Phos (95 mg, 0.20 mmol), Pd₂(dba)₃ (183 mg, 0.20 mmol), and cesium carbonate (3.16 g, 10.00 mmol) were dissolved in 1,4-dioxane (10 ml), and the mixture was heated to 100° C. and stirred for 3 h under nitrogen atmosphere. The reaction solution was cooled, poured into water (10 ml), and extracted with ethyl acetate (10 ml), the organic layer was washed with brine (10 ml), dried over anhydrous sodium sulfate, filtered, and concentrated under reduced pressure to give a crude product, which was purified by column chromatography (PE:EA=8:1) to give 1.6 g of yellow oily product.

Synthesis of Compound 10c: To a solution of Compound 10b (1.6 g, 5.21 mmol) in DCM (10 ml) was added TFA (5 ml), then the reaction mixture was stirred at room temperature for 2 h. The reaction mixture was diluted with ethyl acetate (200 ml), neutralized by saturated sodium bicarbonate aqueous solution (200 ml) to pH 7-8 and layered; the organic layer was dried over anhydrous sodium sulfate, filtered, and concentrated under reduced pressure to give a crude product, which was purified by column chromatography (DCM:MeOH=40:1) to give 970 mg of yellow oily product.

Synthesis of Compound 10d: To a solution of a weighted Compound 5 (200 mg, 0.46 mmol) in 1,4-dioxane (3 ml) was added Compound 10c (95 mg, 0.46 mmol) and DBU (210 mg, 1.38 mmol), then the reaction mixture was heated to 100° C. and stirred for 1 h. The reaction mixture was cooled, poured into water (100 ml), and extracted with ethyl acetate (100 ml), the organic layer was washed with brine (100 ml), dried over anhydrous sodium sulfate, filtered, and concentrated under reduced pressure to give a crude product, which was purified by column chromatography preparation plate (PE:EA=1:1) to give 82 mg of white solid.

Synthesis of Compound I-24: To a solution of Compound 10d (82 mg, 0.14 mmol) in THF (1 ml) was added 0.5M sodium hydroxide aqueous solution (1.0 ml, 0.56 mmol), then the reaction mixture was stirred at room temperature for 2 h. The reaction solution was neutralized with dilute hydrochloric acid to pH 5-6, concentrated under reduced pressure to remove THF and water to give a crude product, the crude product was dissolved with DCM:MeOH=10: 1 (50 ml), and filtered to remove the insoluble matter, the filtrate was concentrated and purified by column chromatography to give 66 mg of white solid. LCMS ESI m/z: 588.2 (M+H)⁺; ¹H NMR (400 MHz, Chloroform-d): δ 7.85 (d, J=8.0 Hz, 1H), 7.53-7.39 (m, 1H), 7.12-6.86 (m, 3H), 6.82 (t, J=8.0 Hz, 1H), 6.27-6.21 (m, 1H), 5.94-5.82 (m, 1H), 4.93-4.80 (m, 1H), 4.18-3.90 (m, 2H), 3.87 (s, 3H), 3.75 (s, 6H), 3.68-3.57 (m, 1H), 3.44 (d, J=12.0 Hz, 1H), 3.31-3.14 (m, 2H), 3.05-2.86 (m, 2H).

Synthesis of Compound I-49: To a solution of Compound I-24 (51 mg, 0.09 mmol) in acetone (2 ml) was added cesium carbonate (88 mg, 0.27 mmol) and bromomethyl acetate (21 mg, 0.14 mmol), then the reaction mixture was heated to 56° C. and stirred for 1 h. The reaction mixture was poured into water (50 ml), and extracted twice with ethyl acetate (50 ml), the organic layers were dried over anhydrous sodium sulfate, filtered, and concentrated under reduced pressure to give a crude product, which was purified by column chromatography preparation plate (DCM:MeOH=20:1) to give 13 mg of white solid. LCMS ESI m/z: 660.2 (M+H)⁺; ¹H NMR (400 MHz, Chloroform-d): δ 7.85 (d, J=16.0 Hz, 1H), 7.43 (t, J=8.0 Hz, 1H), 7.06-6.99 (in, 2H), 6.94-6.89 (in, 1H), 6.78 (d, J=4.0 Hz, 1H), 6.30-6.26 (in, 1H), 5.96-5.87 (in, 1H), 5.80-5.79 (in, 1H), 5.70-5.68 (in, 1H), 5.40-5.20 (in, 2H), 4.87-4.80 (in, 1H), 4.13 (s, 1H), 3.88 (s, 3H), 3.77 (s, 3H), 3.47 (d, J=9.4 Hz, 1H), 3.32-2.93 (in, 4H), 2.73-2.67 (in, 1H), 2.10 (d, J=8.0 Hz, 3H), 2.06-1.92 (in, 1H).

Using the same synthesis process, the following compounds were synthesized and chirally separated:

| Compound ID | Compound structure | LCMS ESI m/z |
|---|---|---|
| I-41 | 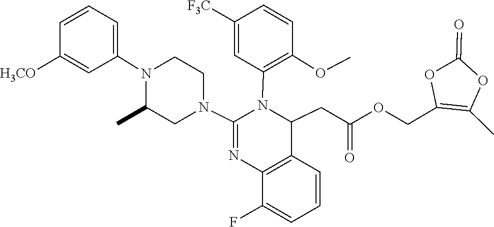 [SMILES: COc1cccc(N2CCN(C3=Nc4c(F)cccc4C(CC(=O)OCc4oc(=O)oc4C)N3c3cc(C(F)(F)F)ccc3OC)C[C@H]2C)c1] | 699.2 |
| I-25 | 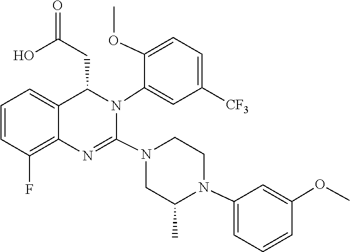 [SMILES: COc1cccc(N2CCN(C3=Nc4c(F)cccc4[C@H](CC(=O)O)N3c3cc(C(F)(F)F)ccc3OC)C[C@H]2C)c1] | 587.3 |
| I-26 | 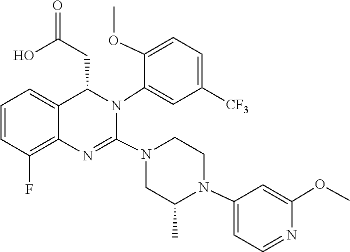 [SMILES: COc1cc(N2CCN(C3=Nc4c(F)cccc4[C@H](CC(=O)O)N3c3cc(C(F)(F)F)ccc3OC)C[C@H]2C)ccn1] | 588.2 |
| I-33 | 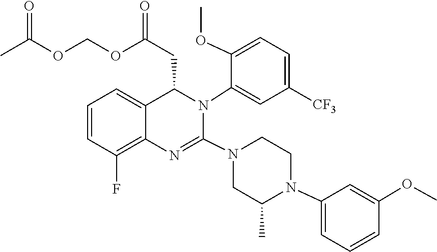 [SMILES: COc1cccc(N2CCN(C3=Nc4c(F)cccc4[C@H](CC(=O)OCOC(C)=O)N3c3cc(C(F)(F)F)ccc3OC)C[C@H]2C)c1] | 659.3 |
| I-53 | 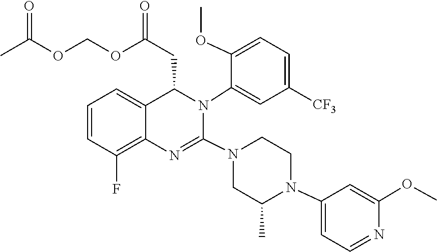 [SMILES: COc1cc(N2CCN(C3=Nc4c(F)cccc4[C@H](CC(=O)OCOC(C)=O)N3c3cc(C(F)(F)F)ccc3OC)C[C@H]2C)ccn1] | 660.2 |

### Embodiment 8: In Vitro Biological Activity Study

Compounds to be tested: Compounds of the present disclosure: Compound I-5, Compound I-8, Compound I-13, Compound I-17, Compound I-19, Compound I-23, Compound I-24, Compound I-25, Compound I-26, Compound I-29, Compound I-33, Compound I-41, Compound I-49, Compound I-53; control compound: letermovir (racemic) (self-synthesized, racemates of unseparated enantiomers).

Experimental method for in vitro biological activity study: The HCMV virus used in this experiment contains a reporter gene GFP. The inhibitory activity of the compounds against HCMV was detected by detecting the expression of GFP, and the cytotoxicity was also determined; the compounds were tested at 8 concentrations, with 3-fold gradient dilution, and double wells. MRC5 cells were seeded in microtiter plates at a certain density and cultured overnight at 37° C. in 5% CO₂. The compounds and viruses were added the next day. Based on the fact that terminal enzyme inhibitors can only inhibit the release and reinfection of progeny viruses and have no inhibitory activity against the initially added HCMV viruses, a 100% inhibition virus control group (cells infected with the virus and added with 1 p M letermovir) and a virus infection control group (cells infected with the virus without compound treatment) were set. The final concentration of DMSO in the cell culture medium was 0.5%, the cells were cultured at 37° C. in 5% CO₂ for 7 days, the fluorescence value of each well was detected using Acumen, and the original data were used to calculate the antiviral activity of the compounds. The cytotoxicity experiment was the same as the antiviral experiment, but without viral infection; CCK-8 reagent was used to detect cell viability, and the original data were used to calculate the cytotoxicity of the compounds. Software GraphPad Prism was used to analyze the dose-response curves of the compounds and calculate the EC₅₀ and CC₅₀ values (see Table 1 for the results).

TABLE 1
The inhibitory activity of compounds against HCMV

| CPD ID | EC₅₀ (nM) | CC₅₀ (μM) |
|---|---|---|
| 1-5 | 2.62 | >50 |
| I-8 | 1.47 | >50 |
| I-13 | 2.76 | >50 |
| I-17 | 1.52 | 43.49 |
| I-19 | 2.81 | 23.76 |
| I-23 | 0.28 | >50 |
| I-24 | 0.46 | >50 |
| I-25 | 0.17 | >50 |
| I-26 | 0.26 | >50 |
| I-29 | 0.10 | 37.14 |
| I-33 | 0.06 | >50 |
| I-41 | 0.15 | 39.28 |
| I-49 | 0.46 | >50 |
| I-53 | 0.32 | >50 |
| Letermovir (racemic) | 3.92 | 36.48 |

Conclusion: Compared with letermovir, the above-mentioned compounds of the present disclosure have more potent activity in inhibiting HCMV, especially Compounds I-23, I-24, I-25, I-26, I-29, I-33, I-41, I-49 and I-53, which have activity below 1 nM, and have compelling clinical application prospects.

### Embodiment 9: Hepatocyte Metabolic Stability Test

Compound to be tested: Compound of the present disclosure: Compound I-25; control compound: letermovir (its structural formula is

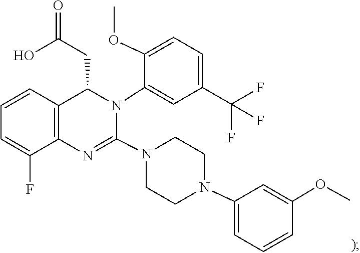 [SMILES: COc1cccc(N2CCN(C3=Nc4c(F)cccc4[C@H](CC(=O)O)N3c3cc(C(F)(F)F)ccc3OC)CC2)c1]

positive control compounds: 7-ethoxycoumarin, 7-hydroxycoumarin.

Test method: The compounds to be tested will be co-incubated with human hepatocyte suspension at a concentration of 1 μM. At different time points, ice acetonitrile containing internal standard was added to the system to terminate the reaction, after vortexing and centrifugation, the supernatant was taken, and the test compounds in the supernatant were analyzed by LC-MS/MS method. The in vitro intrinsic clearance rate was calculated according to the clearance rate constant of the tested compounds in the incubation system. 7-ethoxycoumarin and 7-hydroxycoumarin were used as positive controls, and the blank culture medium incubation group without hepatocytes was used as negative control. The incubation conditions are summarized in Table 2 below:

TABLE 2
Incubation conditions of hepatocyte metabolic stability test

| Hepatocytes | 1.0 × 10⁶ | cells/mL |
|---|---|---|
| Incubating medium | Williams' Medium E | |
| Tested compounds | 1 | μM |
| Positive control | 3 | μM |
| Final volume of | 50 | μL |
| incubation system | | |
| Incubating time | 0, 5, 15, 30, 60, 90 | min (Tested compounds) |
| | 0, 90 | min (Negative control) |
| | 0, 5, 15, 30, 60, 90 | min (Positive control) |

| | |
|---|---|
| Parallel reaction | Parallel 2 copies |

Data analysis: The ratio of the peak areas of the analyte/internal standard (A_{analyte}/A_{IS}) was obtained by the instrument, and the remaining percentage (% Control) was calculated from the ratio of A_{analyste}/A_{IS} in the sample at non-zero time point to that at the zero time point. Ln (% Control) was plotted against incubation time and linearly fitted. The clearance constant (k, min⁻¹), clearance half-life (T_{1/2}, min), and in vitro intrinsic clearance rate (CL_{int, hepatocyte}, μL/min/10⁶ cells) of the tested compounds were calculated using the following equation.

$$k = - slope$$

$${T}_{1 / 2} = 0.693 / k$$

$$V = Incubation volume / Number of incubated cells ( \times {10}^{6} )$$

$${CL}_{int , hepatocyte} = kV$$

$${CL}_{int , hepatic} ( mL / min / kg ) = {CL}_{int , hepatocyte} ( μL / min / {10}^{6} cells ) \times Number of cells per gram of liver weight ( {10}^{6} cells / g liver weight ) \times liver weight ( g liver / kg weight )$$

CL_{int,hepatic} (mL/min/kg) refers to the inherent clearance rate of liver in vivo. The related physiological parameters are shown in table 3 below:

TABLE 3
Related physiological parameters

| | Hepatocytes | Liver |
|---|---|---|
| Species | 10⁶ cells/g liver weight | g liver/kg weight |
| Human | 120 | 21 |

Reference: Natilie A. Hosea and Wendy T. Collard (2009), J Clin Pharmacol, 49; 513-533; Sohlenius-Sternbeck A K (2006) Toxicol. In Vitro 20; 1582-1586; Davies B. and Morris T. (1993), Pharma Res 10(7); 1093-1095.

The test results obtained are shown in table 4 below:

TABLE 4
Results of the hepatocyte metabolic stability test of the compounds

| | | | | Cl_{int, hepatocyte} | CL_{int, hepatic} |
|---|---|---|---|---|---|
| Compound | Species | k (1/min) | T_{1/2} (min) | (μL/min/10⁶ cells) | (mL/min/kg) |
| I-25 | Human | 0.000154 | 4496 | 0.154 | 0.388 |
| Letermovir | Human | 0.00220 | 316 | 2.20 | 5.53 |
| 7-ethoxycoumarin | Human | 0.0348 | 19.9 | 34.8 | 87.7 |
| 7-hydroxycoumarin | Human | 0.0401 | 17.3 | 40.1 | 101 |

It can be seen that Compound I-25 of this application has better metabolic stability of human hepatocytes than letermovir.

The explanation on the above embodiments is only to help understanding of the method and its core concept of the present disclosure. It should be noted that, for those ordinary skilled in the art, various improvements and modifications can be made without depart from the technical principle of the present disclosure, and these improvements and modifications should be covered by the protective scope of the present disclosure.

1. (canceled)

2. A heterocyclic derivative represented by formula (I), or a stereoisomer, a pharmaceutically acceptable salt thereof, 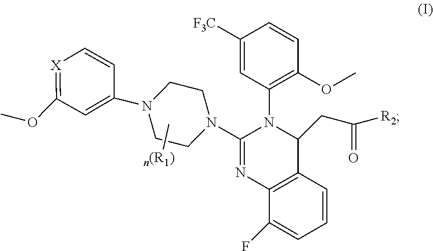
wherein:
R₁ is independently selected from C₁₋₆ alkyl, or two independent R₁ together with the different carbons to which they are attached on the piperazine form a bridged ring;
n is selected from 0, 1, or 2;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy;
X is selected from N or —CH—;
When n is 0 and X is —CH—, R₂ is not hydroxyl.

3. The heterocyclic derivative or the stereoisomer, the pharmaceutically acceptable salt thereof according to claim 2, wherein, the heterocyclic derivative is represented by the following formula (I-a): 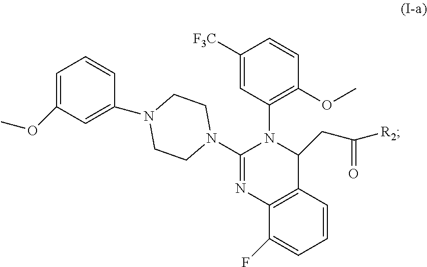
wherein:
R₂ is selected from C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy

4. The heterocyclic derivative or the stereoisomer, the pharmaceutically acceptable salt thereof according to claim 2, wherein, the heterocyclic derivative is represented by the following formula (I-b): 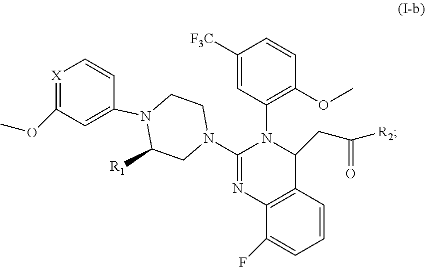
wherein:
R₁ is selected from C₁₋₆ alkyl;
X is selected from N or —CH—;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy.

5. The heterocyclic derivative or the stereoisomer, the pharmaceutically acceptable salt thereof according to claim 2, wherein, the heterocyclic derivative is represented by the following formula (I-c): 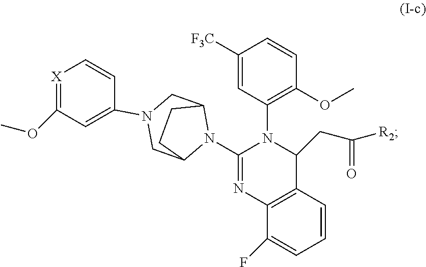
wherein:
X is selected from N or —CH—;
R₂ is selected from hydroxyl, C₁₋₆ hydrocarbyl carbonyloxy C₁₋₆ hydrocarbyloxy, C₃₋₆ cycloalkyl carbonyloxy C₁₋₆ hydrocarbyloxy, C₁₋₆ hydrocarbyloxy carbonyloxy C₁₋₆ hydrocarbyloxy, C₃₋₆ cycloalkoxy carbonyloxy C₁₋₆ hydrocarbyloxy, or substituted or unsubstituted C₄₋₆ heterocycloalkyl C₁₋₆ hydrocarbyloxy.

6. The heterocyclic derivative or the stereoisomer, the pharmaceutically acceptable salt thereof according to claim 2, wherein, R₁ is selected from methyl, ethyl, or isopropyl.

7. The heterocyclic derivative or the stereoisomer, the pharmaceutically acceptable salt thereof according to claim 2, wherein, R₂ is selected from

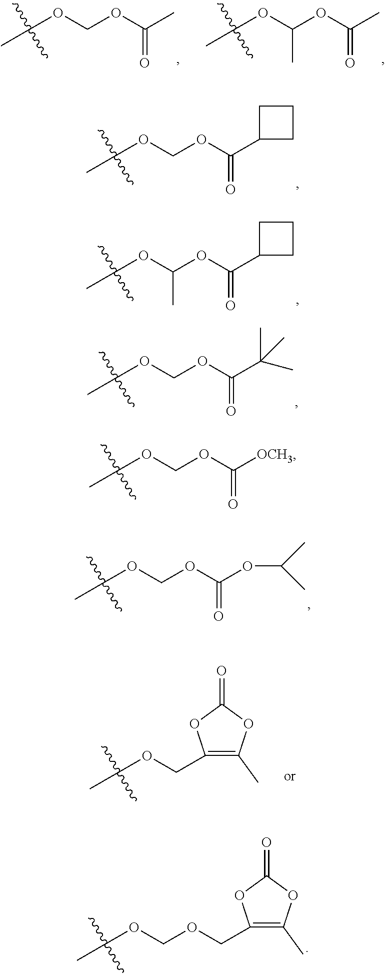

8. The heterocyclic derivative or the stereoisomer, the pharmaceutically acceptable salt thereof according to claim 3, wherein, R₂ is selected from

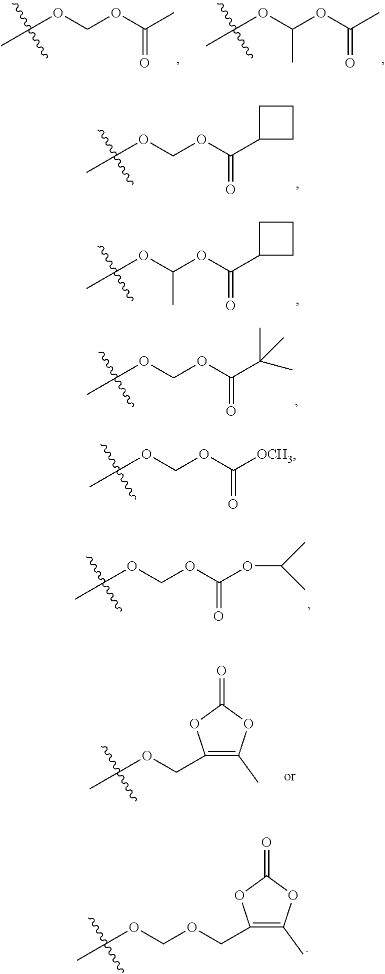

9. The heterocyclic derivative or the stereoisomer, the pharmaceutically acceptable salt thereof according to claim 4, wherein, R₂ is selected from hydroxyl,

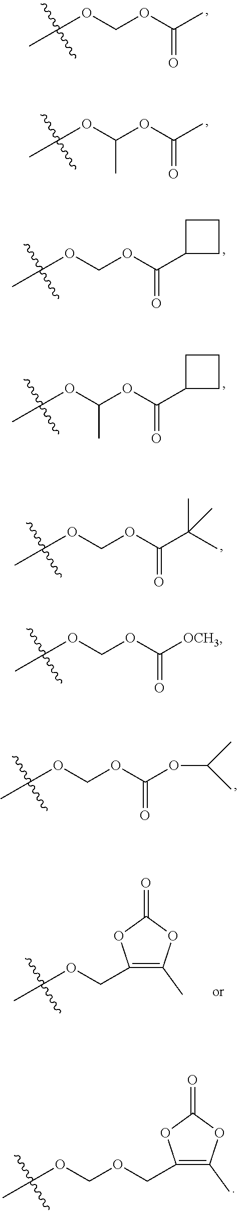

10. The heterocyclic derivative or the stereoisomer, the pharmaceutically acceptable salt thereof according to claim 2, wherein, the heterocyclic derivative or its stereoisomer is selected from the following compounds:

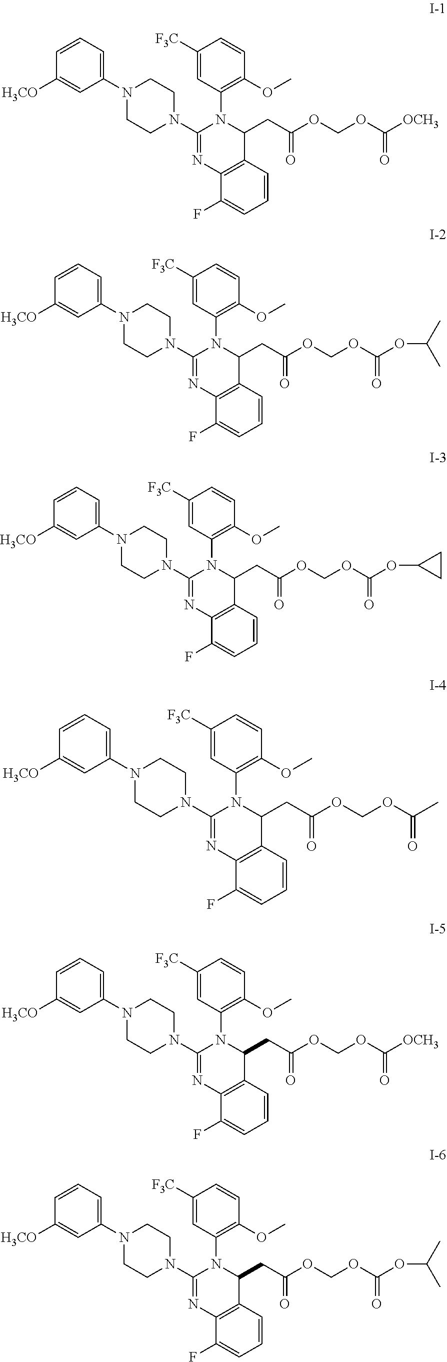

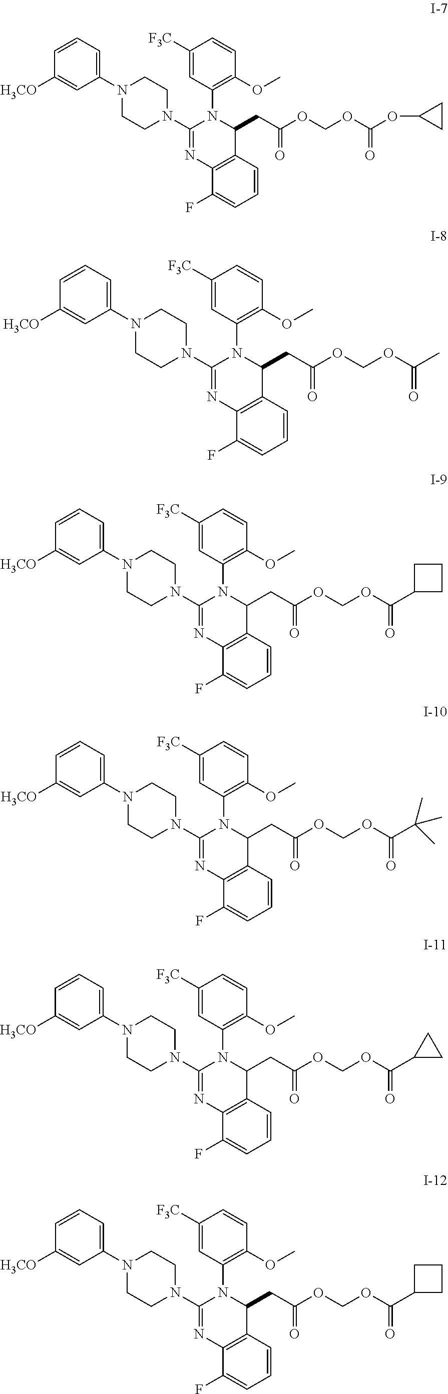

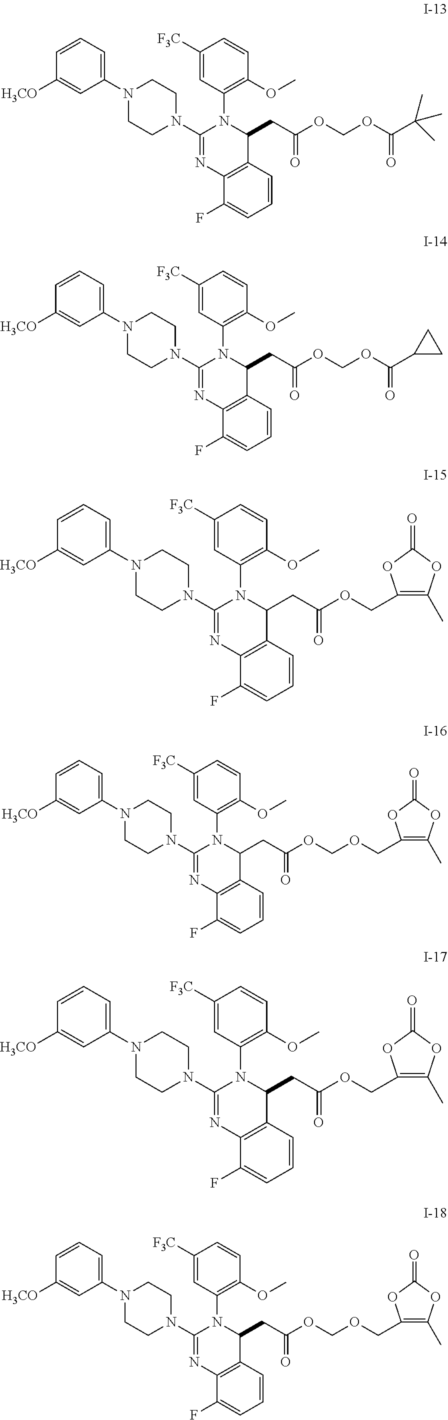

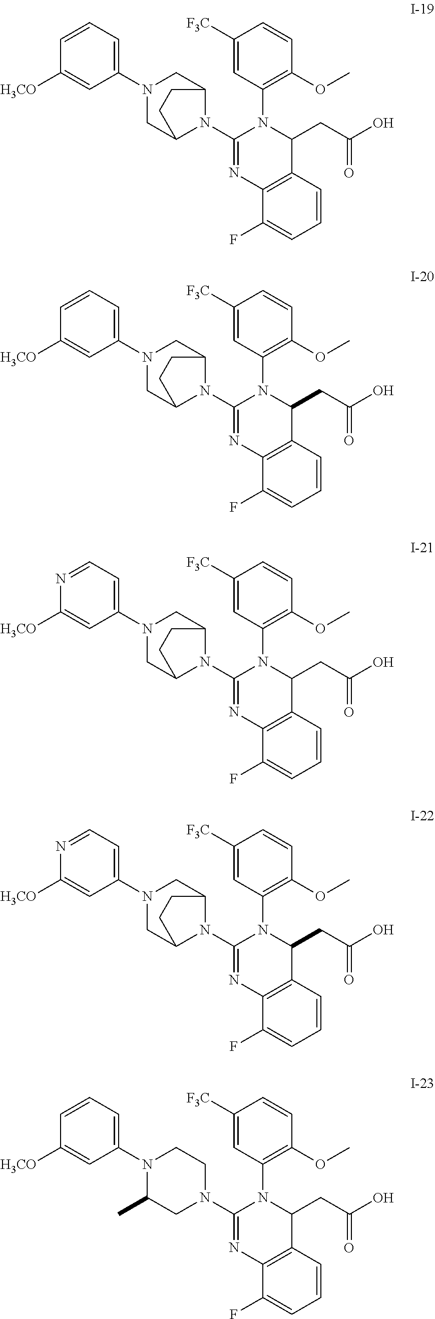 [SMILES: COc1cccc(N2CC3CCC(C2)N3C2=Nc3c(F)cccc3C(CC(=O)O)N2c2cc(C(F)(F)F)ccc2OC)c1 ; COc1cccc(N2CC3CCC(C2)N3C2=Nc3c(F)cccc3[C@H](CC(=O)O)N2c2cc(C(F)(F)F)ccc2OC)c1 ; COc1cc(N2CC3CCC(C2)N3C2=Nc3c(F)cccc3C(CC(=O)O)N2c2cc(C(F)(F)F)ccc2OC)ccn1 ; COc1cc(N2CC3CCC(C2)N3C2=Nc3c(F)cccc3[C@H](CC(=O)O)N2c2cc(C(F)(F)F)ccc2OC)ccn1 ; COc1cccc(N2CCN(C3=Nc4c(F)cccc4C(CC(=O)O)N3c3cc(C(F)(F)F)ccc3OC)C[C@H]2C)c1]

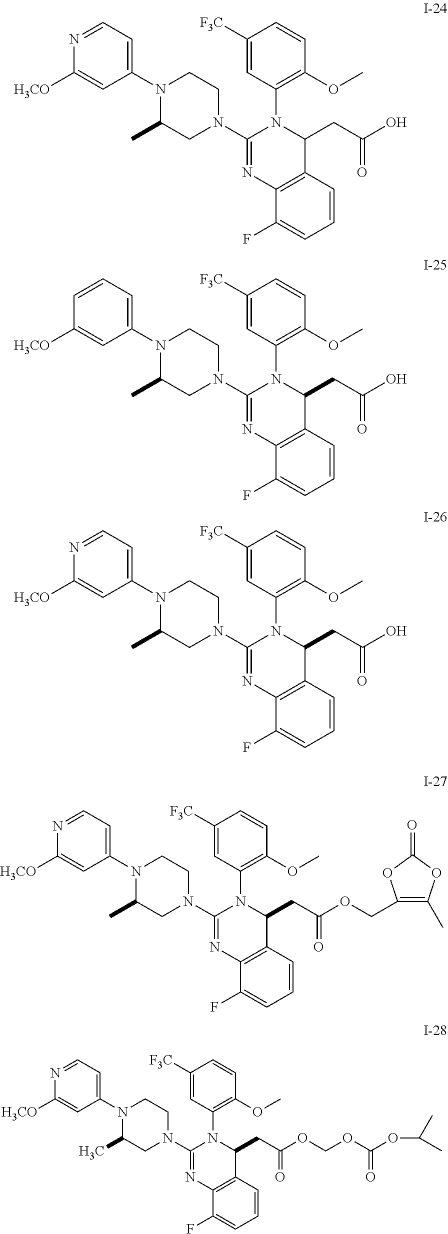

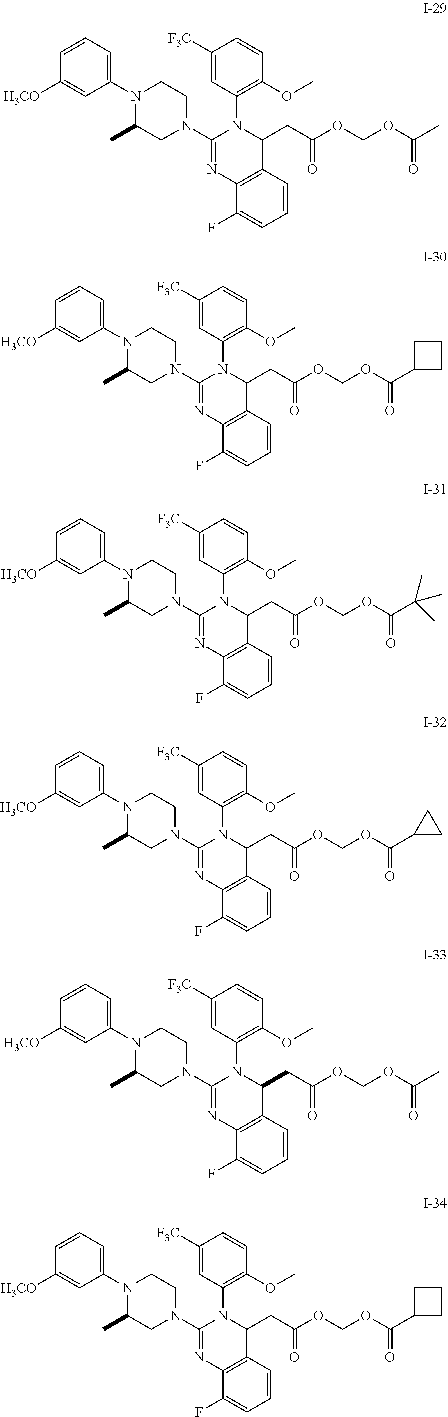

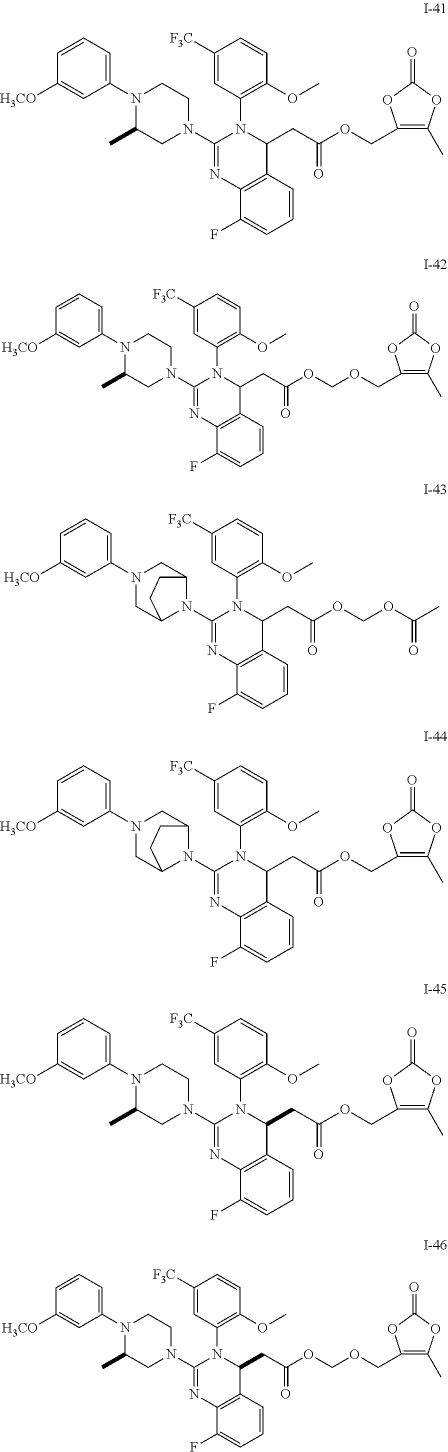

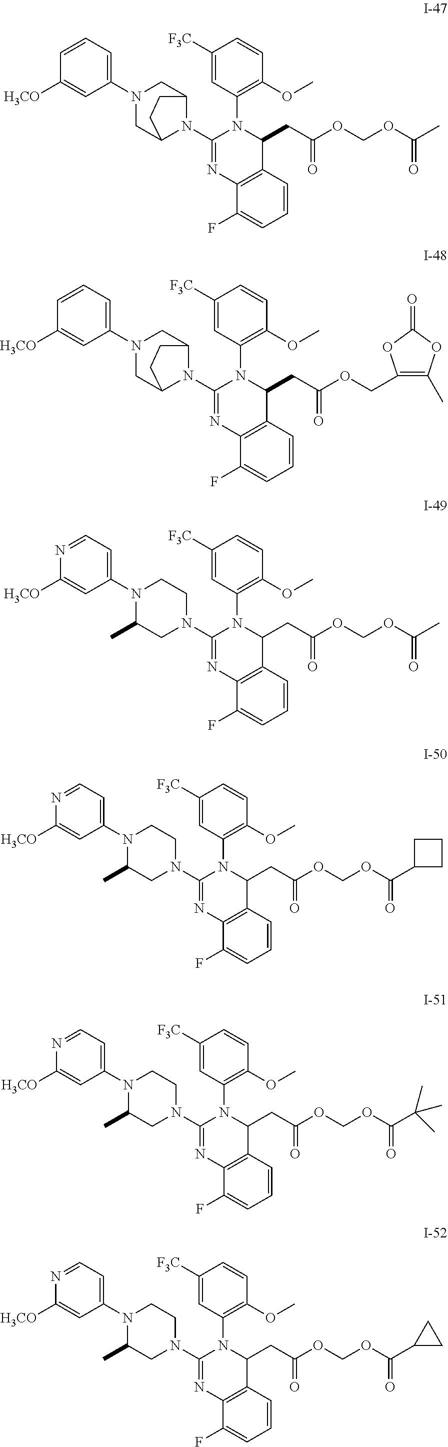

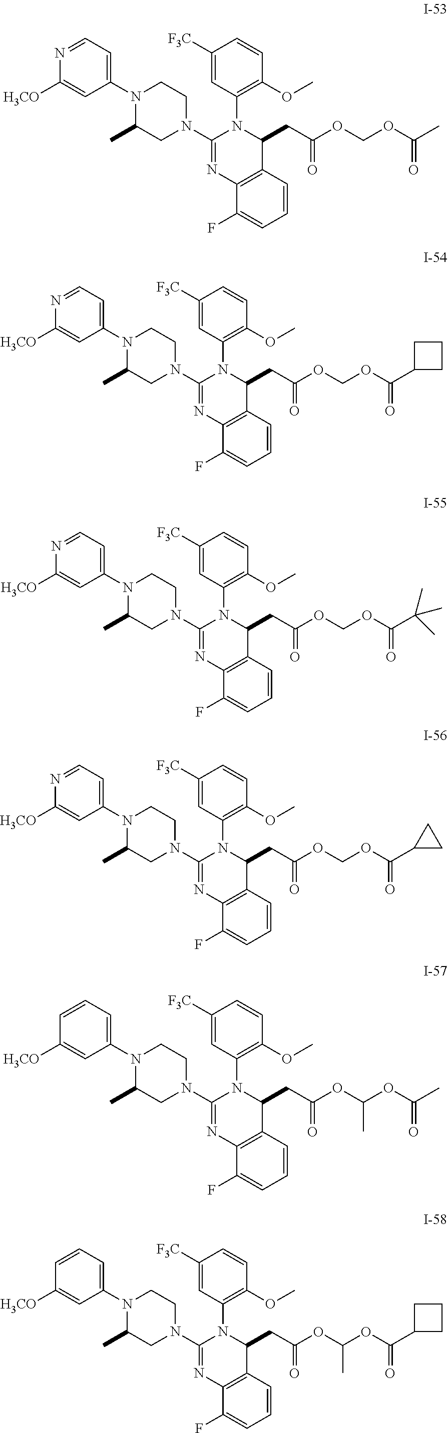

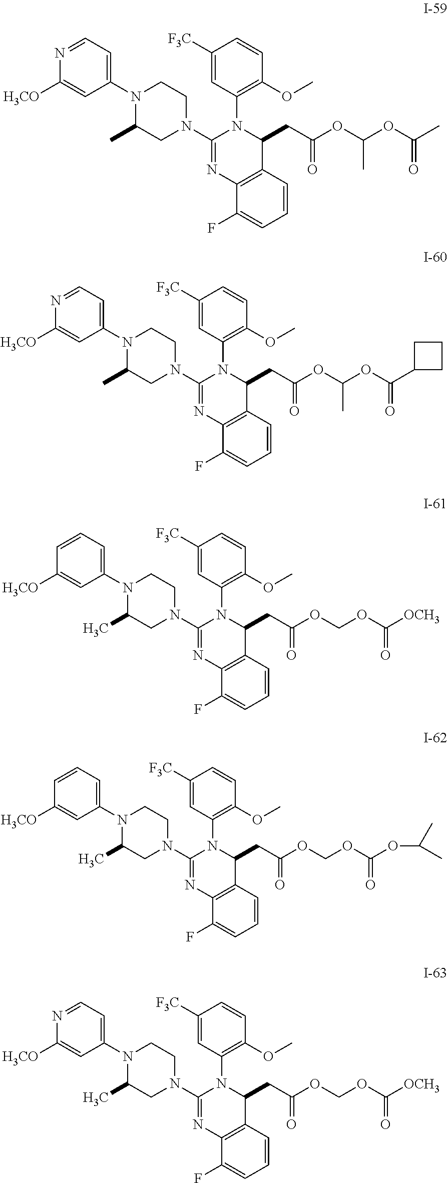

11. A pharmaceutical composition, wherein, it comprises one or more heterocyclic derivatives or stereoisomers, pharmaceutically acceptable salts thereof according to claim 2, and a pharmaceutically acceptable carrier.

12. The pharmaceutical composition according to claim 11, herein, the pharmaceutical composition further comprises one or more therapeutic agents or preventive drugs selected from the group consisting of vaccines, antibody drugs, antibody-drug conjugates, nucleoside drugs, other drugs against human cytomegalovirus infection, and combinations thereof.

13-14. (canceled)

15. The heterocyclic derivative or the stereoisomer, the pharmaceutically acceptable salt thereof according to claim 2, wherein, the heterocyclic derivative is represented by the following formula (I-a) or formula (I-b) or formula (I-c): 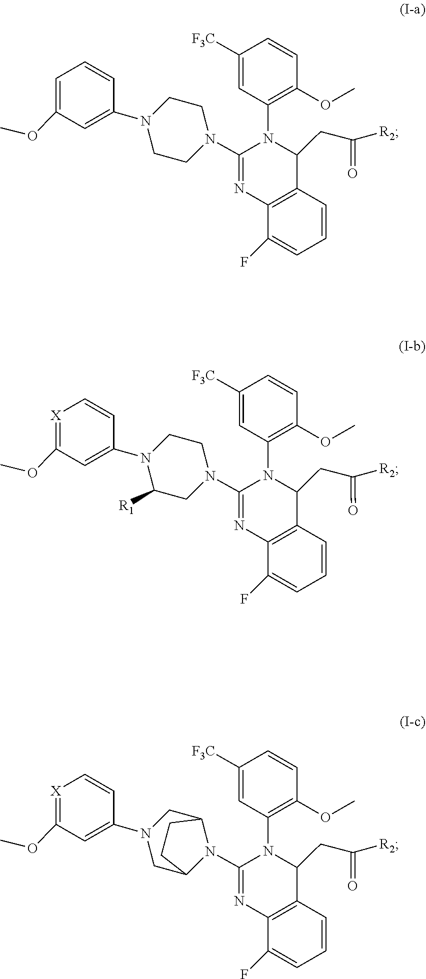
wherein
R₁ is selected from methyl, ethyl, or isopropyl;
R₂ is selected from hydroxyl, 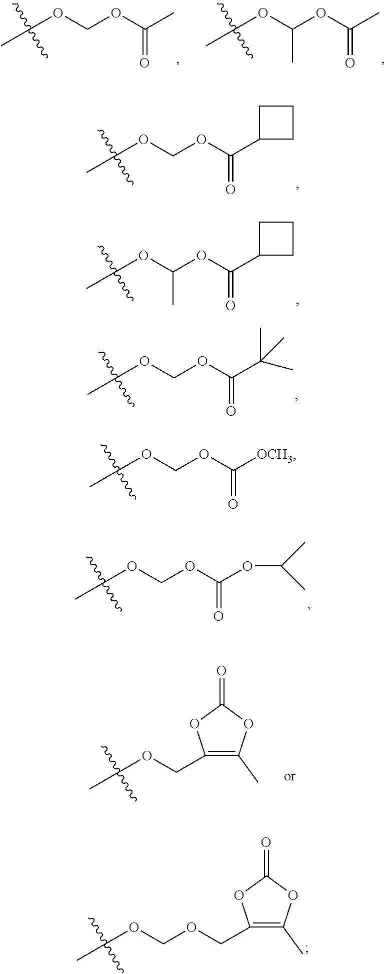
X is selected from N or —CH—;
and in formula (I-a), R₂ is not hydroxyl.

16. A method for prophylaxis and/or treatment of cytomegalovirus infection, wherein, the method comprises administering to a human subject in need of the treatment an effective amount of heterocyclic derivative or the stereoisomer, the pharmaceutically acceptable salt thereof according to claim 2.

17. The method for prophylaxis and/or treatment of cytomegalovirus infection according to claim 15, wherein, the human subjects is an adult CMV-seropositive recipient [R+] of an allogeneic hematopoietic stem cell transplant or adult kidney transplant recipient at high risk, wherein the donor is CMV-seropositive and the recipient is CMV-seronegative [D+/R−].
